(19) United States
(12) Patent Application Publication
(10) Pub. No.: US 2023/0406951 A1
(43) Pub. Date: Dec. 21, 2023

(54) ANTI-TNFR2 ANTIBODIES AND METHODS OF USE THEREOF

(71) Applicant: Pfizer Inc., New York, NY (US)

(72) Inventors: Todd Douglas ASHWORTH, Belmont, MA (US); Yue LIU, Newton, MA (US); Nicholas Andrew MARZE, Qunicy, MA (US); Virginie MCMANUS, Dublin (IE)

(21) Appl. No.: 18/323,629

(22) Filed: May 25, 2023

Related U.S. Application Data
(60) Provisional application No. 63/499,860, filed on May 3, 2023.
(60) Provisional application No. 63/365,371, filed on May 26, 2022.

(51) Int. Cl. C07K 16/28; A61P 37/06

(52) U.S. Cl. CPC C07K 16/2878; A61P 37/06; A61K 2039/505

(57) ABSTRACT

The present disclosure provides antibodies that bind to TNFR2 as well as uses of the antibodies and associated methods. The disclosure also provides processes for making, preparing, and producing antibodies that bind to TNFR2. Antibodies of the disclosure are useful in one or more of diagnosis, prophylaxis, or treatment of disorders or conditions mediated by, or associated with, TNFR2 activity.

### REFERENCE TO SEQUENCE LISTING

“The instant application contains a Sequence Listing which has been submitted electronically in .xml format and is hereby incorporated by reference in its entirety. Said .xml copy, created on May 5, 2023 named PC072865 Sequence Listing.xml and is 42,322 bytes in size.”

### BACKGROUND

The present invention relates to antibodies that specifically bind TNFR2 and compositions, methods and uses thereof, including use of antibodies of the disclosure to treat autoimmunity and autoinflammation diseases. The present invention also pertains to related molecules, e.g. nucleic acids which encode such antibodies compositions, and related methods, e.g., methods for producing and purifying such antibodies and bispecific antibodies, and their use in diagnostics and therapeutics.

TNF is a pleiotropic cytokine expressed on the cell surface of leukocytes and subsequently released through the activity of several proteolytic enzymes. Cellular responses to TNF are mediated by two receptors, TNFR1 (TNFRSF1A), which is ubiquitously expressed and mediates pro-inflammatory responses, and TNFR2 (TNFRSF1B) which is more selectively expressed on specific leukocyte subtypes and seems to mediate predominately immunoregulatory effects (Salomon 2021). Soluble TNF can activate both receptors, but TNFR2 is more preferentially activated by membrane associated TNF. Amongst the activities attributed to TNFR2 agonism, the most relevant for therapy of autoimmune and autoinflammatory conditions are expansion of regulatory T cells, activation induced cell death and exhaustion of effector T cells, and enhancement of regulatory/anti-inflammatory phenotypes in B cells, mesenchymal stem cells (MSC) and myeloid cells such as myeloid derived suppressor cells (MDSC) and glia (Faustman and Davis 2010, Salomon 2021).

### SUMMARY

The present disclosure provides antibodies that bind to TNFR2 as well as uses of the antibodies and associated methods. The disclosure also provides processes for making, preparing, and producing antibodies that bind to TNFR2. Antibodies of the disclosure are useful in one or more of diagnosis, prophylaxis, or treatment of disorders or conditions mediated by, or associated with, TNFR2 activity, including, but not limited to rheumatoid arthritis (RA),

ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE). The disclosure further encompasses expression of antibodies, and preparation and manufacture of compositions comprising antibodies of the disclosure, such as medicaments for the use of the antibodies.

Polynucleotides encoding antibodies that bind TNFR2 are provided. Polynucleotides encoding antibody heavy chains or light chains, or both are also provided. Host cells that express the antibodies are provided. Methods of treatment using the antibodies are provided. Such methods include, but are not limited to, one or more of methods of treating or methods of preventing diseases associated with or mediated by TNFR2 expression and or TNFR2 binding rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE)].

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:

E1. An isolated antibody that specifically binds to TNFR2, comprising a heavy chain variable region (VH) and a light chain variable region (VL), comprising the CDR-H1, CDR-H2, and CDR-H3 sequences of a VH sequence selected from the group consisting of SEQ ID NO: 13, SEQ ID NO: 19, SEQ ID NO: 21, SEQ ID NO: 22, SEQ ID NO: 29, and SEQ ID NO: 30; and the CDR-L1, CDR-L2, and CDR-L3 sequences of a VL sequence selected from the group consisting of SEQ ID NO: 4, SEQ ID NO: 6, SEQ ID NO: 8, and SEQ ID NO: 9.

E2. An isolated antibody that specifically binds to TNFR2, comprising a heavy chain variable region (VH) and a light chain variable region (VL), comprising the CDR-H1, CDR-H2, and CDR-H3 sequences according to SEQ ID NO: 30, and the CDR-L1, CDR-L2, and CDR-L3 sequences according to SEQ ID NO: 9.
E3. An isolated antibody that specifically binds to TNFR2, comprising a heavy chain variable region (VH) and a light chain variable region (VL), comprising
(i) a CDR-L1 sequence according to SEQ ID NO: 1; a CDR-L2 sequence according to SEQ ID NO: 7, and a CDR-L3 sequence according to SEQ ID NO: 3, and a CDR-H1 sequence according to SEQ ID NO: 10; a CDR-H2 sequence according to SEQ ID NO: 20; a CDR-H3 sequence according to SEQ ID NO: 12; or
(ii) a CDR-L1 sequence according to SEQ ID NO: 1; a CDR-L2 sequence according to SEQ ID NO: 2, and a CDR-L3 sequence according to SEQ ID NO: 3, and a CDR-H1 sequence according to SEQ ID NO: 10; a CDR-H2 sequence according to SEQ ID NO: 11; a CDR-H3 sequence according to SEQ ID NO: 12.
   E4. An isolated antibody that specifically binds to TNFR2, comprising a heavy chain variable region (VH) and a light chain variable region (VL), wherein a CDR-L1 sequence according to SEQ ID NO: 1; a CDR-L2 sequence according to SEQ ID NO: 7, and a CDR-L3 sequence according to SEQ ID NO: 3, and wherein a CDR-H1 sequence according to SEQ ID NO: 10; a CDR-H2 sequence according to SEQ ID NO: 20; a CDR-H3 sequence according to SEQ ID NO: 12.
   E5. The antibody of any one of E1-E4, comprising a VH framework sequence derived from a human germline VH sequence selected from the group consisting of IGHV1-46, IGHV4-31, IGHV4-30-4, and IGHV4-4.
   E6. The antibody of any one of E1-E5, comprising a VH framework sequence derived from a human IGHV1-46 germline sequence.
   E7. The antibody of any one of E1-E6, comprising a VL framework sequence derived from a human germline VL sequence selected from the group consisting of IGKV1-9, IGKV1-33, IGKV1-27, IGKV1-39, IGKV1-9, IGKV1-1, and IGKV1-11.
   E8. The antibody of any one of E1-E7, comprising a VL framework sequence derived from a human germline IGKV1-9 sequence.
   E9. The antibody of any one of E1-E8, comprising a VL framework sequence and a VH framework sequence wherein one or both of the VL framework sequence and the VH framework sequence is at least 90%, 91%, 92%, 93%, 94%, 95%, 96%, 97%, 98%, 99%, or 100% identical to the human germline sequence from which it was derived.
   E10. The antibody of any one of E1-E9, comprising a VL framework sequence and a VH framework sequence, and wherein one or both of the VL framework sequence or the VH framework sequence is identical to the human germline sequence from which it was derived.
   E11. The antibody of any one of E1-E10, wherein the VL comprises an amino acid sequence according to a sequence selected from the group consisting of SEQ ID NO: 4 and SEQ ID NO: 8, and wherein the VH comprises an amino acid sequence according to a sequence selected from the group consisting of SEQ ID NO: 13 and SEQ ID NO: 21.
   E12. The antibody of any one of E1-E11, comprising the VH sequence of SEQ ID NO: 13, and the VL of SEQ ID NO: 4.
   E13. The antibody of any one of E1-E11, comprising a VH sequence at least 90%, 91%, 92%, 93%, 94%, 95%, 96%, 97%, 98%, 99%, or 100% identical to SEQ ID NO: 21, and comprising a VL sequence at least 90%, 91%, 92%, 93%, 94%, 95%, 96%, 97%, 98%, 99%, or 100% identical to SEQ ID NO: 8.
   E14. The antibody of any one of E13, comprising the VH sequence of SEQ ID NO: 21, and the VL of SEQ ID NO: 8.
   E15. The antibody of any one of E14, comprising a VH sequence encoded by a nucleic acid sequence of SEQ ID NO: 32.
   E16. The antibody of any one of E14, comprising a VH sequence encoded by a nucleic acid sequence of SEQ ID NO: 33.
   E17. The antibody of any one of E1-E16, comprising a VL sequence encoded by a nucleic acid sequence of SEQ ID NO: 31.
   E18. The antibody of any one of E1-E17, comprising one or both of a VH sequence encoded by the plasmid deposited at the ATCC and having ATCC Accession No. PTA-127528 and a VH sequence encoded by the plasmid deposited at the ATCC and having ATCC Accession No. PTA-127529.
   E19. The antibody of any one of E1-E18, comprising a VL sequence encoded by the plasmid deposited at the ATCC and having ATCC Accession No. PTA-127531.
   E20. An antibody comprising one or both of a VH sequence encoded by the plasmid deposited at the ATCC and having ATCC Accession No. PTA-127528 and a VH sequence encoded by the plasmid deposited at the ATCC and having ATCC Accession No. PTA-127529; and a VL sequence encoded by the plasmid deposited at the ATCC and having ATCC Accession No. PTA-127531.
   E25. The antibody of E1-E24, further comprising a constant heavy domain (CH1) and a constant light domain (CL).
   E26. The antibody of E25, wherein the CH1 is connected to the VH, and the CL is connected to the VL forming a Fab domain.
   E26. The antibody of E25, comprising a first and a second Fab domain.
   E27. The antibody of any one of E25-E26, comprising an antibody Fc domain comprising a first Fc chain and a second Fc chain.
   E28. The antibody of E27, wherein the first Fab domain is covalently fused to the first Fc chain, and the second Fab domain is covalently fused to the second Fc chain.
   E29. The antibody of E27-E28, wherein the C-terminus of the CH1 domain in the first Fab domain is covalently fused to the N-terminus of the first Fc chain, and the C-terminus of the CH1 domain in the second Fab domain is covalently fused to the N-terminus of the second Fc chain.
   E30. The antibody of E31, wherein the Fc domain is the Fc domain of an IgA (for example IgA₁, or IgA₂), IgD, IgE, IgM, or IgG (for example IgG₁, IgG₂, IgG₃, or Iga₄).
   E31. The antibody of E30, wherein the Fc domain is the Fc domain of an IgG₁.
   E32. The antibody of E31, wherein the CH1 domain in the first Fab domain comprises a sequence according to SEQ ID NO: 23.
   E33. The antibody of E31-E32, wherein the CL in the first Fab domain comprises a sequence according to SEQ ID NO: 5.
   E34. The antibody of E31-E33, wherein the antibody comprises a light chain (LC) comprising a sequence in accordance with SEQ ID NO: 9.
   E35. The antibody of E31-E34, wherein the First Fab domain and the second Fab domain are identical.
   E36. The antibody of E31-E35, wherein the first Fc chain comprises, from N-terminus to C-terminus: a first hinge region, a first CH2 region, and a first CH3 region, and the second Fc chain comprises, from N-terminus to C-terminus: a second hinge region, a second CH2 region, and a second CH3 region.
   E37. The antibody of E36, wherein one or both of the first hinge region and second hinge region comprises a sequence according to SEQ ID NO: 23.
   E38. The antibody of E36-E37, wherein one or both of the first CH2 domain and second CH2 domain comprises a sequence according to SEQ ID NO: 25.
   E39. The antibody of E36-E38, wherein one or both of the first CH3 domain and second CH3 domain comprises a sequence according to SEQ ID NO: 26.
   E40. The antibody of E36-E39, wherein one or both of the first Fc chain and second Fc chain comprises a sequence according to SEQ ID NO: 37.
   E41. The antibody of E36-E40, wherein the first Fc chain and the second Fc chain are identical.
   E42. The antibody of E32-E41, wherein the antibody comprises a heavy chain (HC) comprising a sequence in accordance with SEQ ID NO: 22.
   E43. The antibody of E26-E42, further comprising a third Fab and a fourth Fab.
   E44. The antibody of E43, wherein the first Fab, second Fab, third Fab, and fourth Fab each comprise a CDR-L1 sequence according to SEQ ID NO: 1; a CDR-L2 sequence according to SEQ ID NO: 7, and a CDR-L3 sequence according to SEQ ID NO: 3, and a CDR-H1 sequence according to SEQ ID NO: 10; a CDR-H2 sequence according to SEQ ID NO: 20; a CDR-H3 sequence according to SEQ ID NO: 12.
   E45. The antibody of E43-E44, wherein the first Fab, second Fab, third Fab, and fourth Fab each comprise a VH with a sequence according to SEQ ID NO: 21 and a VL with a sequence according to SEQ ID NO: 8.
   E46. The antibody of E43-E45, wherein the first Fab, second Fab, third Fab, and fourth Fab are identical to each other.
   E47. The antibody of E43-E46, wherein the N′ terminus of the first Fab is connected to the C′-terminus of the third Fab.
   E48. The antibody of E43-E47, wherein the N′ terminus of the first Fab is connected to the C′-terminus of the third Fab via a first linker.
   E49. The antibody of E48, wherein the first linker comprises a sequence in accordance with SEQ ID NO: 27.
   E50. The antibody of E43-E49, wherein the N′ terminus of the second Fab is connected to the C′-terminus of the fourth Fab.
   E51. The antibody of E43-E50, wherein the N′ terminus of the second Fab is connected to the C′-terminus of the fourth Fab via a second linker.
   E52. The antibody of E51, wherein the second linker comprises a sequence in accordance with SEQ ID NO: 27.
   E53. The antibody of E47-E48, wherein the HC comprises a sequence according to SEQ ID NO: 30.
   E54. An isolated antibody that specifically binds to TNFR2, comprising a heavy chain (HC) comprising a sequence according to SEQ ID NO: 30, and a light chain (LC), comprising a sequence according to SEQ ID NO: 9.
   E55. The antibody of any one of E1-E54, comprising a VH sequence encoded by a nucleic acid sequence of SEQ ID NO: 33.
   E56. The antibody of any one of E1-E55, comprising a VL sequence encoded by a nucleic acid sequence of SEQ ID NO: 31.
   E57. An isolated antibody that specifically binds to TNFR2, comprising a heavy chain (HC) sequence encoded by a nucleic acid sequence of SEQ ID NO: 33, and a light chain (LC) sequence encoded by a nucleic acid sequence of SEQ ID NO: 31.
   E58. The antibody of any one of E1-E57, comprising a HC sequence encoded by the plasmid deposited at the ATCC and having ATCC Accession No. PTA-127530.
   E59. The antibody of any one of E1-E58, comprising a LC sequence encoded by the plasmid deposited at the ATCC and having ATCC Accession No. PTA127532.
   E60. An antibody comprising a HC sequence encoded by the plasmid deposited at the ATCC and having ATCC Accession No. PTA-127530 and a LC sequence encoded by the plasmid deposited at the ATCC and having ATCC Accession No. PTA-127532.
   E61. The antibody of E30, wherein the Fc domain is the Fc domain of an IgG₂.
   E62. The antibody of E61, wherein the CH1 domain in the first Fab domain comprises a sequence according to SEQ ID NO: 14.
   E63. The antibody of E61-E62, wherein the CL in the first Fab domain comprises a sequence according to SEQ ID NO: 5.
   E64. The antibody of E61-E63, wherein the First Fab domain and the second Fab domain are identical.
   E65. The antibody of E61-E64, wherein the first Fc chain comprises, from N-terminus to C-terminus: a first hinge region, a first CH2 region, and a first CH3 region, and the second Fc chain comprises, from N-terminus to C-terminus: a second hinge region, a second CH2 region, and a second CH3 region.
   E66. The antibody of E65, wherein one or both of the first hinge region and second hinge region comprises a sequence according to SEQ ID NO: 15.
   E67. The antibody of E65-E66, wherein one or both of the first CH2 domain and second CH2 domain comprises a sequence according to SEQ ID NO: 16.
   E68. The antibody of E65-E67, wherein one or both of the first CH3 domain and second CH3 domain comprises a sequence according to SEQ ID NO: 17.
   E69. The antibody of E65-E68, wherein one or both of the first Fc chain and second Fc chain comprises a sequence according to SEQ ID NO: 18.
   E70. The antibody of E65-E69, wherein the first Fc chain and the second Fc chain are identical.
   E71. The antibody of E75-E70, wherein the antibody comprises a heavy chain (HC) comprising a sequence in accordance with SEQ ID NO: 19.
   E72. The antibody of E65-E71, wherein the antibody comprises a light chain (LC) comprising a sequence in accordance with a sequence selected from the group consisting of SEQ ID NO: 6, and SEQ ID NO: 9.
   E73. The antibody of E65-E72, wherein the antibody comprises a light chain (LC) comprising a sequence in accordance with SEQ ID NO: 9.
   E74. An isolated antibody that specifically binds to TNFR2, comprising a heavy chain (HC) comprising a sequence in accordance with SEQ ID NO: 19, and a light chain (LC) comprising a sequence in accordance with SEQ ID NO: 9.
   E75. The antibody of E1-E74, wherein the antibody is characterized by an EC₅₀ of less than 5 μM in a human TNFR2 potency assay in Jurkat reporter cells.
   E76. The antibody of E1-E75, wherein the antibody is characterized by an EC₅₀ of less than 2 μM in a human TNFR2 potency assay in Jurkat reporter cells.
   E77. The antibody of E1-E76, wherein the antibody is characterized by an EC₅₀ of less than in a human TNFR2 potency assay in human peripheral blood monocytes.
   E78. The antibody of E1-E77, wherein the antibody is characterized by an EC₅₀ of less than in a human TNFR2 potency assay in human peripheral blood monocytes.
   E79. The antibody of E1-E78, wherein the antibody is characterized by an EC₅₀ of less than 2 μM in a human TNFR2 potency assay in human peripheral blood monocytes.
   E80. The antibody of E1-E79, wherein the antibody is characterized by an EC₅₀ of less than 20 μM in a cynomolgus TNFR2 potency assay in cynomolgus peripheral blood monocytes.
   E81. The antibody of E1-E80, wherein the antibody is characterized by an EC₅₀ of less than in a cynomolgusTNFR2 potency assay in cynomolgus peripheral blood monocytes.
   E82. The antibody of E1-E81, wherein the antibody is characterized by an EC₅₀ of less than in a human TNFR2 potency assay in Jurkat reporter cells.
   E83. The antibody of E1-E82, wherein the antibody is characterized by an EC₅₀ of less than 1 μM in a human TNFR2 potency assay in Jurkat reporter cells.
   E84. The antibody of E1-E83, wherein the antibody is characterized by an EC₅₀ of less than in a human TNFR2 potency assay in Jurkat reporter cells.
   E85. The antibody of E1-E84, wherein the antibody is characterized by an EC50 of less than 2 mg/ml for ICAM-1 upregulation in human TNFR2 expressing primary T-cell population from human peripheral blood monocytes.
   E86. The antibody of E1-E85, wherein the antibody is characterized by an EC50 of less than for ICAM-1 upregulation in cynomolgus TNFR2 expressing primary T-cell population from cynomolgus peripheral blood monocytes.
   E87. The antibody of E1-E86, wherein the antibody is characterized by an EC50 of less than 1 mg/mi for ICAM-1 upregulation in human TNFR2 expressing primary T-cell population from human peripheral blood monocytes.
   E88. The antibody of E1-E87, wherein the antibody is characterized by an EC50 of less than 0.2 mg/ml for ICAM-1 upregulation in human TNFR2 expressing primary T-cell population from human peripheral blood monocytes.
   E89. the antibody of E1-E88, wherein the antibody is characterized by an EC50 of less than for ICAM-1 upregulation in cynomolgus TNFR2 expressing primary T-cell population from cynomolgus peripheral blood monocytes.
   E90. The antibody of E1-E89, wherein the antibody is characterized by an affinity KD for human TNFR2 of less than 1 nm.
   E91. The antibody of E1-E90, wherein the antibody is characterized by an affinity KD for human TNFR2 of less than 0.5 nm.
   E92. The antibody of E1-E91, wherein the antibody is characterized by an affinity KD for human TNFR2 of less than 0.1 nm.
   E93. The antibody of E1-E92, wherein the antibody is characterized by an affinity KD for human TNFR2 of less than 0.07 nm.
   E94. The antibody of E1-E93, wherein the antibody is characterized by an affinity KD for cynomolgus TNFR2 of less than 1 nm.
   E95. The antibody of E1-E94, wherein the antibody is characterized by an affinity KD for cynomolgus TNFR2 of less than 0.1 nm.
   E96. An isolated antibody comprising the VH and VL of an antibody selected from Table 35.
   E97. The antibody of any one of E1-99, for use as a medicament.
   E98. The antibody of E97, wherein the use is for the treatment of one or more selected from the group consisting of rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE).
   E99. The antibody of any one of E96-E98, wherein the use is for rheumatoid arthritis (RA).
   E100. An isolated polynucleotide, comprising one or more nucleotide sequences encoding the antibody of any one of E1-E99.
   E101. The polynucleotide of E100, wherein said polynucleotide is RNA.
   E102. The polynucleotide of E100-E101, wherein said polynucleotide comprises at least one chemical modification.
   E103. The polynucleotide of E102, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine and 2′-O-methyl uridine.
   E104. The polynucleotide of E100-E101, wherein said polynucleotide does not comprise a chemical modification.
   E105. An isolated polynucleotide encoding a HC and a LC, or both, of an antibody that binds to TNFR2, wherein said nucleic acid comprises one or more selected from the group consisting of the nucleic acid sequence of SEQ ID NO: 31, the nucleic acid sequence of SEQ ID NO: 32, the nucleic acid sequence of SEQ ID NO: 33.
   E106. An isolated polynucleotide encoding an isolated antibody that specifically binds to TNFR2, comprising a heavy chain (HC) sequence encoded by a nucleic acid sequence of SEQ ID NO: 33, and a light chain (LC) sequence encoded by a nucleic acid sequence of SEQ ID NO: 31
   E107. An isolated polynucleotide encoding an isolated antibody comprising a HC sequence encoded by the plasmid deposited at the ATCC and having ATCC Accession No. PTA-127530 and a LC sequence encoded by the plasmid deposited at the ATCC and having ATCC Accession No. PTA-127532.
   E108. A vector comprising the polynucleotide of E100-E107.
   E109. An isolated host cell comprising the polynucleotide of E100-E107, or the vector of E108.
   E110. A method of producing an isolated antibody, comprising culturing the host cell of E109 under conditions that result in production of the antibody, and recovering the antibody.
   E111. A pharmaceutical composition comprising a therapeutically effective amount of the antibody of E1-E99 and a pharmaceutically acceptable carrier.
   E112. A method of treating a medical condition, comprising administering to a subject in need thereof a therapeutically effective amount of the antibody of any one of E1-E99, the polynucleotide of E101, or the pharmaceutical composition of E111.
   E113. The method of E112, wherein the condition is selected from the group consisting of rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE).
   E114. The method of any one of E112-E113, comprising administering said antibody or pharmaceutical composition, subcutaneously.
   E115. The method of any one of E112-E114 wherein said antibody thereof, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.

### FIGURES

FIG. 1: analytical SEC profile of the commercially available MR2-1 clone and the 1-step purified clone 162

FIG. 2: representation of the structure of a TetraFab, showing the extra Fab domain labelled outer VH-outer CH1) added on the inner VH (labelled inner VH-inner CH1). The outer and inner VHs are linked by a G4S domain.

FIG. 3 Survival curve for TetraFab-2053 vs Control lg. Statistics: Log-rank (Mantel-Cox) test. Data also shown in Table 21.

FIG. 4: Body Weight percentage change versus baseline. Data also shown in Table 22 FIG. 5: Mean plasma human IFNγ (pg/ml). *** p<0.001 TF2053 vs Control lg. Data also shown in Table 23.

FIG. 6: Mean plasma human IL10 (pg/ml). *** p<0.001 TF2053 vs Control lg. Data also shown in Table 24.

FIG. 7: Mean plasma human IL17A (pg/ml). ** p<0.01 TF2053 vs Control lg. *** p<0.001 TF2053 vs Control lg. Data also shown in Table 25.

FIG. 8: Mean plasma human TNFα (pg/ml). *** p<0.001 TF2053 vs Control lg. Statistics: unpaired 2-tailed Student's t-Test. Data also shown in FIG Table 26.

FIG. 9: Mean liver weight per body weight at sacrifice (mg/kg). *** p<0.001 TF2053 vs Control Ig. Data also shown in Table 27.

FIG. 10: Mean spleen weight per body weight at sacrifice (mg/kg). *** p<0.001 TF2053 vs Control lg. Statistics: unpaired 2-tailed Student's t-Test. Data also shown in Table 28.

FIG. 11: Mean percent human CD45+ cells in whole blood leukocytes. ** p<0.01. *** p<0.001. **** p<0.0001. Data also shown in Table 29.

FIG. 12: Mean percent human CD4+ of human CD45+/CD3+ whole blood lymphocytes. **** p<Data also shown in Table 30.

FIG. 13: Mean percent human CD8+ of human CD45+/CD3+ whole blood lymphocytes. **** p<Data also shown in Table 31.

FIG. 14: Mean percent anergic (PD1+KLRG1−CD57−) cells of human CD8+/CD3+/CD45+ blood lymphocytes. **** p<0.0001. Data also shown in Table 32.

FIG. 15: Mean percent exhausted (PD1+KLRG1+CD57−) cells of human CD8+/CD3+/CD45+ blood lymphocytes. ** p<0.01. Data also shown in Table 33.

FIG. 16: Mean percent senescent (PD1-KLRG1+CD57+) cells of human CD8+/CD3+/CD45+ blood lymphocytes. * p<0.05. ** p<0.01. **** p<0.0001. Statistics: unpaired 2-tailed T test between groups at each timepoint. Data also shown in Table 34.

### DETAILED DESCRIPTION

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of ***) means that the parameter may vary by as much as 10% below or above the stated numerical value for that parameter. For example, a dose of about 5 mg means 5%±10%, i.e. it may vary between 4.5 mg and 5.5 mg.

### Antibody

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific), and includes portions of intact antibodies that retain the ability to bind to a given antigen (e.g. an “antigen-binding fragment”), and any other modified configuration of an immunoglobulin molecule that comprises an antigen binding site.

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

### Variable Region

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions.

### Constant Region

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL)

### Fc Domain and Fc Chain

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification

### Monoclonal Antibody

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

### Human Antibody

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

### Chimeric Antibody

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

### Humanized Antibody

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

### Antigen

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

### Epitope

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level the epitope can be characterized by the amino acid residues that it comprises as defined by a specific criterion, e.g., by distance between atoms (e.g., heavy, i.e., non-hydrogen atoms) in the antibody and the antigen. At a further less detailed level the epitope can be characterized through function, e.g., by competition binding with other antibodies. The epitope can also be defined more generically as comprising amino acid residues for which substitution by another amino acid will alter the characteristics of the interaction between the antibody and antigen (e.g. using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

### Binding Affinity

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K D). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K D is the ratio of the rate of dissociation, also called the “off-rate (k off)” or “k d” to the association rate, or “on-rate (kₒₙ)” or “kₐ”. Thus, K_{D} equals k_{off}/kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K D values for antibodies can be determined using methods well established in the art. One exemplary method for determining the K D of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K D of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

### Monospecific Antibody

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

### Bispecific Antibody

A “bispecific antibody” refers to a molecule that has binding specificity for at least two different epitopes. In some embodiments, bispecific antibodies can bind simultaneously two different antigens. In other embodiments, the two different epitopes may reside on the same antigen.

### Tetrafab

A tetrafab molecule refers to an antibody or antigen binding portion thereof comprising four antigen binding sites. The antigen binding sites may bind one, two, three, or four different epitopes, and such epitopes may be on one, two, three, or four different targets.

### Half Maximal Effective Concentration (EC₅₀)

The term “half maximal effective concentration (EC₅₀)” refers to the concentration of a therapeutic agent which causes a response halfway between the baseline and maximum after a specified exposure time. The therapeutic agent may cause inhibition or stimulation. The EC₅₀ value is commonly used, and is used herein, as a measure of potency.

### Agonist

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

### Antagonist

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

### Compete

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

### Fc Receptor

An “Fc receptor” (FcR) refers to a receptor that binds to the Fc region of an antibody. In some embodiments, an FcR is a native human FcR. In some embodiments, an FcR is one which binds an IgG antibody (a gamma receptor) and includes receptors of the FcgRI, FcgRII, and FcgRIII subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptor tyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol 1991; 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward., Immunol. Today 1997; 18(12):592-598; Ghetie et al., Nature Biotechnology, 1997; 15(7):637-640; Hinton et al., J. Biol. Chem. 2004; 279(8):6213-6216; WO 2004/92219).

### Effector Cells

An “effector cell” refers to a leukocyte which express one or more FcRs and performs effector functions. In certain embodiments, effector cells express at least FcgRIII and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

### Antibody-Dependent Cell-Mediated Cytotoxicity (ADCC)

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII. To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc region amino acid sequences and increased or decreased ADCC activity are described, e.g., in U.S. Pat. Nos. 7,923,538, and 7,994,290.

### Enhanced ADCC Activity

The term “enhanced ADCC activity” refers to an antibody that is more effective at mediating ADCC in vitro or in vivo compared to the parent antibody, wherein the antibody and the parent antibody differ in at least one structural aspect, and when the amounts of such antibody and parent antibody used in the assay are essentially the same. In some embodiments, the antibody and the parent antibody have the same amino acid sequence, but the antibody is afucosylated while the parent antibody is fucosylated. In some embodiments, ADCC activity will be determined using an in vitro ADCC assay, but other assays or methods for determining ADCC activity, e.g. in an animal model etc., are contemplated. In some embodiments, an antibody with enhanced ADCC activity has enhanced affinity for FcgRIIIA.

### Altered FcR Binding or ADCC Activity

The term “altered” FcR binding affinity or ADCC activity refers to an antibody which has either enhanced or diminished activity for one or more of FcR binding activity or ADCC activity compared to a parent antibody, wherein the antibody and the parent antibody differ in at least one structural aspect. An antibody that “displays increased binding” to an FcR binds at least one FcR with better affinity than the parent antibody. An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc region.

### Complement Dependent Cytotoxicity (CDC)

The term “Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 1996; 202: 163, may be performed. Antibodies with altered Fc region amino acid sequences and increased or decreased C1q binding capability are described, e.g., in U.S. Pat. Nos. 6,194,551, 7,923,538, 7,994,290 and WO 1999/51642.

### Host Cell

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

### Vector

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

### Isolated

An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

### Polypeptide/Protein

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

### Polynucleotide/Nucleic Acid

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

### Conservative Substitution

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine.

### Identity

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

### Excipient

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient” “includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

### Treating

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is X.

### Prevent

The terms “prevent” or “prevention” refer to one or more of delay of onset, reduction in frequency, or reduction in severity of at least one sign or symptom (e.g., ***specific for particular application) of a particular disease, disorder or condition (e.g., ***). In some embodiments, prevention is assessed on a population basis such that an agent is considered to “prevent” a particular disease, disorder or condition if a statistically significant decrease in the development, frequency or intensity of one or more symptoms of the disease, disorder or condition is observed in a population susceptible to the disease, disorder or condition. Prevention may be considered complete when onset of disease, disorder or condition has been delayed for a predefined period of time.

### Subject

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development. In some embodiments, a subject is a patient with disease rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE).

### Therapeutically Effective Amount

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

### Antibodies to TNFR2

The disclosure provides antibodies that bind to TNFR2, also known as tumor necrosis factor receptor superfamily member 1B (TNFRSF1B), CD120b or p75. TNFR2 is one of two membrane receptors that binds tumor necrosis factor-alpha (TNFα), the other being TNFR1. TNFR2 is a type I transmembrane receptor, with an extracellular domain made of 4 cysteine-rich domains CRD. Binding of TNFα to TNFR2 triggers an intracellular signaling cascade leading to NF-KB activation and subsequently cell proliferation, activation and survival.]

As used herein, the term TNFR2 includes variants, isoforms, homologs, orthologs and paralogs of TNFR2. In some embodiments, an antibody disclosed herein cross-reacts with TNFR2 from species other than human, such as TNFR2 of cynomolgus monkey, as well as different forms of TNFR2. In some embodiments, an antibody may be completely specific for human TNFR2 and may not exhibit species cross-reactivity (e.g., does not bind mouse TNFR2) or other types of cross-reactivity. As used herein TNFR2 refers to naturally occurring human TNFR2 unless contextually dictated otherwise. Therefore, a “TNFR2 antibody” “anti-TNFR2 antibody” or other similar designation means any antibody (as defined herein) that binds or reacts with TNFR2, an isoform, fragment or derivative thereof. The full length, mature form of TNFR2, as represented by UniProtKB/Swiss-Prot accession number P20333 is herein provided as SEQ ID NO: 34. The full length, mature form of mouse TNFR2, as represented by UniProtKB/Swiss-Prot accession number P25119 is herein provided as SEQ ID NO: 35. The full length, mature form of cynomolgus TNFR2, as represented by NCBI database accession number XP 005544817 is herein provided as SEQ ID NO: 36.

In some aspects, the antibodies of the disclosure agonize TNFR2. In some aspects, antibodies of the disclosure do not inhibit TNFα binding to the TNFR2 molecule bound by the antibody.

In some embodiments, an anti-TNFR2 antibody of the disclosure encompasses an antibody that one or both of i) competes for binding to human TNFR2 with or ii) binds the same epitope as, an antibody having the amino acid sequence of a heavy chain variable region set forth as SEQ ID NO: 21 and the amino acid sequence of a light chain variable region set forth as SEQ ID NO: 8.

Anti-TNFR2 antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, an anti-TNFR2 antibody is a monoclonal antibody. In some embodiments, an anti-TNFR2 antibody is a human or humanized antibody. In some embodiments, an anti-TNFR2 antibody is a chimeric antibody.

In some embodiments, the invention provides an antibody having a light chain variable region (VL) sequence and a heavy chain variable region (VH) sequence as found in the sequence list table herein, or variants thereof.

The invention also provides CDR portions of antibodies to TNFR2. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the antibody comprises one or both of i) the full-length heavy chain, with or without the C-terminal lysine, or ii) the full-length light chain of anti-TNFR2 antibody TF-2053.

In certain embodiments, an antibody described herein comprises an Fc domain. The Fc domain can be derived from IgA (e.g., IgA, or IgA 2), IgG, IgE, or IgG (e.g., IgG₁, IgG₂, IgG₃, or IgG₄). In some embodiments, an anti-TNFR2antibody is an IgG1antibody.

The invention encompasses modifications to the CDRs and variable regions shown in Table 5. For example, the invention includes antibodies comprising functionally equivalent variable regions and CDRs which do not significantly affect their properties as well as variants which have enhanced or decreased activity or affinity. For example, the amino acid sequence may be mutated to obtain an antibody with the desired binding affinity to TNFR2. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

In some aspects, the disclosure provides an isolated antibody that specifically binds to TNFR2, comprising a heavy chain variable region (VH) and a light chain variable region (VL), comprising the CDR-H1, CDR-H2, and CDR-H3 sequences of a VH sequence selected from the group consisting of SEQ ID NO: 13, SEQ ID NO: 19, SEQ ID NO: 21, SEQ ID NO: 22, SEQ ID NO: 29, and SEQ ID NO: 30; and the CDR-L1, CDR-L2, and CDR-L3 sequences of a VL sequence selected from the group consisting of SEQ ID NO: 4, SEQ ID NO: 6, SEQ ID NO: 8, and SEQ ID NO: 9.

In some aspects, the disclosure provides an isolated antibody that specifically binds to TNFR2, comprising a heavy chain variable region (VH) and a light chain variable region (VL), comprising the CDR-H1, CDR-H2, and CDR-H3 sequences according to SEQ ID NO: 30, and the CDR-L1, CDR-L2, and CDR-L3 sequences according to SEQ ID NO: 9.

In some aspects, the disclosure provides an isolated antibody that specifically binds to TNFR2, comprising a heavy chain variable region (VH) and a light chain variable region (VL), comprising
(i) a CDR-L1 sequence according to SEQ ID NO: 1; a CDR-L2 sequence according to SEQ ID NO: 7, and a CDR-L3 sequence according to SEQ ID NO: 3, and a CDR-H1 sequence according to SEQ ID NO: 10; a CDR-H2 sequence according to SEQ ID NO: 20; a CDR-H3 sequence according to SEQ ID NO: 12; or
(ii) a CDR-L1 sequence according to SEQ ID NO: 1; a CDR-L2 sequence according to SEQ ID NO: 2, and a CDR-L3 sequence according to SEQ ID NO: 3, and a CDR-H1 sequence according to SEQ ID NO: 10; a CDR-H2 sequence according to SEQ ID NO: 11; a CDR-H3 sequence according to SEQ ID NO: 12.

In some aspects, the disclosure provides an isolated antibody that specifically binds to TNFR2, comprising a heavy chain variable region (VH) and a light chain variable region (VL), wherein a CDR-L1 sequence according to SEQ ID NO: 1; a CDR-L2 sequence according to SEQ ID NO: 7, and a CDR-L3 sequence according to SEQ ID NO: 3, and wherein a CDR-H1 sequence according to SEQ ID NO: 10; a CDR-H2 sequence according to SEQ ID NO: 20; a CDR-H3 sequence according to SEQ ID NO: 12.

The VH framework sequence may be derived from a human germline VH sequence selected from the group consisting of IGHV1-46, IGHV4-31, IGHV4-30-4, and IGHV4-4. In some aspects, the VH framework sequence may derived from a human IGHV1-46 germline sequence.

The VL framework sequence may be derived from a human germline VL sequence selected from the group consisting of IGKV1-9, IGKV1-33, IGKV1-27, IGKV1-39, IGKV1-9, IGKV1-1, and IGKV1-11. In some aspects, the VL framework sequence is derived from a human germline IGKV1-9 sequence.

In some aspects, the disclosure provides an antibody as described comprising a VL framework sequence and a VH framework sequence wherein one or both of the VL framework sequence and the VH framework sequence is at least 90%, 91%, 92%, 93%, 94%, 95%, 96%, 97%, 98%, 99%, or 100% identical to the human germline sequence from which it was derived.

In some aspects, the disclosure provides an antibody as described comprising a VL framework sequence and a VH framework sequence, and wherein one or both of the VL framework sequence or the VH framework sequence is identical to the human germline sequence from which it was derived.

In some aspects, the disclosure provides an antibody wherein the VL comprises an amino acid sequence according to a sequence selected from the group consisting of SEQ ID NO: 4 and SEQ ID NO: 8, and wherein the VH comprises an amino acid sequence according to a sequence selected from the group consisting of SEQ ID NO: 13 and SEQ ID NO: 21.

In some aspects, the disclosure provides an antibody comprising the VH sequence of SEQ ID NO: 13, and the VL of SEQ ID NO: 4.

In some aspects, the disclosure provides an antibody comprising a VH sequence at least 90%, 91%, 92%, 93%, 94%, 95%, 96%, 97%, 98%, 99%, or 100% identical to SEQ ID NO: 21, and comprising a VL sequence at least 90%, 91%, 92%, 93%, 94%, 95%, 96%, 97%, 98%, 99%, or 100% identical to SEQ ID NO: 8.

In some aspects, the disclosure provides an antibody comprising the VH sequence of SEQ ID NO: 21, and the VL of SEQ ID NO: 8.

In some embodiments, the disclosure provides an anti-TNFR2 antibody containing variations of the variable regions shown in the sequence list, the CDRs shown in the sequence list, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in the sequence list. These amounts are not meant to be limiting and increments between the recited percentages are specifically envisioned as part of the disclosure.

In some embodiments, the disclosure provides an anti-TNFR2 antibody comprising a VH sequence encoded by a nucleic acid sequence of SEQ ID NO: 32. In some embodiments, the disclosure provides an anti-TNFR2 antibody comprising a VH sequence encoded by a nucleic acid sequence of SEQ ID NO: 33. In some embodiments, the disclosure provides an anti-TNFR2 antibody comprising a VL sequence encoded by a nucleic acid sequence of SEQ ID NO: 31.

In some embodiments, the disclosure provides an anti-TNFR2 antibody comprising a constant heavy domain (CH1) and a constant light domain (CL). The CH1 may be connected to the VH, and the CL may be connected to the VL forming a Fab domain. The antibody may comprise a first and a second Fab domain.

In some embodiments, the disclosure provides an anti-TNFR2 antibody comprising an antibody Fc domain comprising a first Fc chain and a second Fc chain. The first Fab domain may be covalently fused to the first Fc chain, and the second Fab domain may be covalently fused to the second Fc chain. The C-terminus of the CH1 domain in the first Fab domain may be covalently fused to the N-terminus of the first Fc chain, and the C-terminus of the CH1 domain in the second Fab domain may be covalently fused to the N-terminus of the second Fc chain. The Fc domain may be the Fc domain of an IgA (for example IgA, or IgA 2), IgD, IgE, IgM, or IgG (for example IgG₁, IgG₂, IgG₃, or IgG₄). The Fc domain may be the Fc domain of an IgG₁.

In some embodiments, the disclosure provides an anti-TNFR2 antibody wherein the CH1 domain in the first Fab domain comprises a sequence according to SEQ ID NO: 23.

In some embodiments, the disclosure provides an anti-TNFR2 antibody wherein the CL in the first Fab domain comprises a sequence according to SEQ ID NO: 5.

In some embodiments, the disclosure provides an anti-TNFR2 antibody comprising a light chain (LC) comprising a sequence in accordance with SEQ ID NO: 9.

In some embodiments, the disclosure provides an anti-TNFR2 antibody wherein the First Fab domain and the second Fab domain are identical. The first Fc chain may comprise, from N-terminus to C-terminus: a first hinge region, a first CH2 region, and a first CH3 region, and the second Fc chain may comprise, from N-terminus to C-terminus: a second hinge region, a second CH2 region, and a second CH3 region.

In some embodiments, the disclosure provides an anti-TNFR2 antibody wherein one or both of the first hinge region and second hinge region comprises a sequence according to SEQ ID NO: 23. In some embodiments, the disclosure provides an anti-TNFR2 antibody wherein one or both of the first CH2 domain and second CH2 domain comprises a sequence according to SEQ ID NO: 25. In some embodiments, the disclosure provides an anti-TNFR2 antibody wherein one or both of the first CH3 domain and second CH3 domain comprises a sequence according to SEQ ID NO: 26. In some embodiments, the disclosure provides an anti-TNFR2 antibody wherein one or both of the first Fc chain and second Fc chain comprises a sequence according to SEQ ID NO: 37.

In some embodiments, the disclosure provides an anti-TNFR2 antibody wherein the first Fc chain and the second Fc chain are identical. In some embodiments, the disclosure provides an anti-TNFR2 antibody comprising a heavy chain (HC) comprising a sequence in accordance with SEQ ID NO: 22.

In some embodiments, the disclosure provides an anti-TNFR2 antibody further comprising a third Fab and a fourth Fab.

In some embodiments, the disclosure provides an anti-TNFR2 antibody wherein the first Fab, second Fab, third Fab, and fourth Fab each comprise a CDR-L1 sequence according to SEQ ID NO: 1; a CDR-L2 sequence according to SEQ ID NO: 7, and a CDR-L3 sequence according to SEQ ID NO: 3, and a CDR-H1 sequence according to SEQ ID NO: 10; a CDR-H2 sequence according to SEQ ID NO: 20; a CDR-H3 sequence according to SEQ ID NO: 12.

In some embodiments, the disclosure provides an anti-TNFR2 antibody wherein the first Fab, second Fab, third Fab, and fourth Fab each comprise a VH with a sequence according to SEQ ID NO: 21 and a VL with a sequence according to SEQ ID NO: 8.

In some embodiments, the disclosure provides an anti-TNFR2 antibody wherein the first Fab, second Fab, third Fab, and fourth Fab are identical to each other.

In some embodiments, the disclosure provides an anti-TNFR2 antibody wherein the N′ terminus of the first Fab is connected to the C′-terminus of the third Fab.

In some embodiments, the disclosure provides an anti-TNFR2 antibody wherein the N′ terminus of the first Fab is connected to the C′-terminus of the third Fab via a first linker.

In some embodiments, the disclosure provides an anti-TNFR2 antibody wherein the first linker comprises a sequence in accordance with SEQ ID NO: 27.

In some embodiments, the disclosure provides an anti-TNFR2 antibody wherein the N′ terminus of the second Fab is connected to the C′-terminus of the fourth Fab.

In some embodiments, the disclosure provides an anti-TNFR2 antibody wherein the N′ terminus of the second Fab is connected to the C′-terminus of the fourth Fab via a second linker.

In some embodiments, the disclosure provides an anti-TNFR2 antibody wherein the second linker comprises a sequence in accordance with SEQ ID NO: 27.

In some embodiments, the disclosure provides an anti-TNFR2 antibody wherein the HC comprises a sequence according to SEQ ID NO: 30.
E54. An isolated antibody that specifically binds to TNFR2, comprising a heavy chain (HC) comprising a sequence according to SEQ ID NO: 30, and a light chain (LC), comprising a sequence according to SEQ ID NO: 9.

In some embodiments, the disclosure provides an anti-TNFR2 antibody comprising a VH sequence encoded by a nucleic acid sequence of SEQ ID NO: 33. In some embodiments, the disclosure provides an anti-TNFR2 antibody comprising a VL sequence encoded by a nucleic acid sequence of SEQ ID NO: 31. In some embodiments, the disclosure provides an anti-TNFR2 antibody comprising a heavy chain (HC) sequence encoded by a nucleic acid sequence of SEQ ID NO: 33, and a light chain (LC) sequence encoded by a nucleic acid sequence of SEQ ID NO: 31.

In some embodiments, the disclosure provides an anti-TNFR2 antibody comprising a HC sequence encoded by the plasmid deposited at the ATCC and having ATCC Accession No. PTA-127530. In some embodiments, the disclosure provides an anti-TNFR2 antibody comprising a LC sequence encoded by the plasmid deposited at the ATCC and having ATCC Accession No. PTA127532. In some embodiments, the disclosure provides an anti-TNFR2 antibody comprising a HC sequence encoded by the plasmid deposited at the ATCC and having ATCC Accession No. PTA-127530 and a LC sequence encoded by the plasmid deposited at the ATCC and having ATCC Accession No. PTA-127532.

In some embodiments provided herein is an anti-TNFR2 antibody that comprises a heavy chain and a light chain, wherein the antibody heavy chain has an amino acid sequence encoded by the nucleic acid sequence of the insert of the plasmid deposited with the ATCC having ATCC Accession No. PTA-127530 and the antibody light chain has an amino acid sequence encoded by the nucleic acid sequence of the insert of the plasmid deposited with the ATCC having ATCC Accession No. PTA-127532.

In some embodiments provided herein is an anti-TNFR2 antibody wherein the Fc domain is the Fc domain of an IgG₂. In some embodiments provided herein is an anti-TNFR2 antibody wherein the CH1 domain in the first Fab domain comprises a sequence according to SEQ ID NO: 14. In some embodiments provided herein is an anti-TNFR2 antibody wherein the CL in the first Fab domain comprises a sequence according to SEQ ID NO: 5.

In some embodiments provided herein is an anti-TNFR2 antibody wherein the First Fab domain and the second Fab domain are identical.

In some embodiments provided herein is an anti-TNFR2 antibody wherein the first Fc chain comprises, from N-terminus to C-terminus: a first hinge region, a first CH2 region, and a first CH3 region, and the second Fc chain comprises, from N-terminus to C-terminus: a second hinge region, a second CH2 region, and a second CH3 region.

In some embodiments provided herein is an anti-TNFR2 antibody wherein one or both of the first hinge region and second hinge region comprises a sequence according to SEQ ID NO: In some embodiments provided herein is an anti-TNFR2 antibody wherein one or both of the first CH2 domain and second CH2 domain comprises a sequence according to SEQ ID NO: 16. In some embodiments provided herein is an anti-TNFR2 antibody wherein one or both of the first CH3 domain and second CH3 domain comprises a sequence according to SEQ ID NO: 17. In some embodiments provided herein is an anti-TNFR2 antibody wherein one or both of the first Fc chain and second Fc chain comprises a sequence according to SEQ ID NO: 18.

In some embodiments provided herein is an anti-TNFR2 antibody wherein the first Fc chain and the second Fc chain are identical. In some embodiments provided herein is an anti-TNFR2 antibody wherein the antibody comprises a heavy chain (HC) comprising a sequence in accordance with SEQ ID NO: 19. In some embodiments provided herein is an anti-TNFR2 antibody wherein the antibody comprises a light chain (LC) comprising a sequence in accordance with a sequence selected from the group consisting of SEQ ID NO: 6, and SEQ ID NO: 9.

In some embodiments provided herein is an isolated anti-TNFR2 antibody wherein the antibody comprises a light chain (LC) comprising a sequence in accordance with SEQ ID NO: 9.

In some embodiments provided herein is an isolated antibody that specifically binds to TNFR2, comprising a heavy chain (HC) comprising a sequence in accordance with SEQ ID NO: 19, and a light chain (LC) comprising a sequence in accordance with SEQ ID NO: 9.

The invention also encompasses fusion proteins comprising one or more components of the antibodies disclosed herein. In some embodiments, a fusion protein may be made that comprises all or a portion of an anti-TNFR2 antibody of the invention linked to another polypeptide. In another embodiment, only the variable domains of the anti-TNFR2 antibody are linked to the polypeptide. In another embodiment, the VH domain of an anti-TNFR2 antibody is linked to a first polypeptide, while the VL domain of an anti-TNFR2 antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

### Biological Activity of Anti-TNFR2 Antibodies

In addition to binding an epitope on TNFR2, an antibody of the disclosure can mediate a biological activity. That is, the disclosure includes an isolated antibody that specifically binds TNFR2 and mediates at least one detectable activity selected from the following:
(i) binds specifically to human TNFR2;
(ii) binds specifically to cynomolgus monkey TNFR2;
(iii) stimulates the activity of TNFR2 (e.g., human, cynomolgus); and/or
(iv) permits TNFα activation of TNFR2 while bound to TNFR2

In some embodiments provided herein is an isolated anti-TNFR2 antibody wherein the antibody is characterized by an EC₅₀ in a human TNFR2 potency assay in Jurkat reporter cells of less than a figure selected from group consisting of 5 μM and 2 μM.

In some embodiments provided herein is an isolated anti-TNFR2 antibody wherein the antibody is characterized by an EC₅₀ in a human TNFR2 potency assay in human peripheral blood monocytes of less than a figure selected from group consisting of 10 μM, 5 μM, and 2 μM.

In some embodiments provided herein is an isolated anti-TNFR2 antibody wherein the antibody is characterized by an EC₅₀ in a cynomolgus TNFR2 potency assay in cynomolgus peripheral blood monocytes of less than a figure selected from group consisting of 20 μM and

In some embodiments provided herein is an isolated anti-TNFR2 antibody wherein the antibody is characterized by an EC₅₀ in a human TNFR2 potency assay in Jurkat reporter cells of less than a figure selected from group consisting of 5 μM, 1 μM, and 0.5 μM.

In some embodiments provided herein is an isolated anti-TNFR2 antibody wherein the antibody is characterized by an EC₅₀ for ICAM-1 upregulation in human TNFR2 expressing primary T-cell population from human peripheral blood monocytes of less than a figure selected from group consisting of 10 mg/mi, 5 mg/mi, and 2 mg/mi.

In some embodiments provided herein is an isolated anti-TNFR2 antibody wherein the antibody is characterized by an EC₅₀ for ICAM-1 upregulation in cynomolgus TNFR2 expressing primary T-cell population from cynomolgus peripheral blood monocytes of less than a figure selected from group consisting of 50 mg/mi, 20 mgml, and 15 mg/m, and 10 mg/mi.

In some embodiments provided herein is an isolated anti-TNFR2 antibody wherein the antibody is characterized by an affinity KD for human TNFR2 of less than a figure selected from group consisting of 1 nm, 0.5 nm, 0.1 nm, and 0.07 nm.

In some embodiments provided herein is an isolated anti-TNFR2 antibody wherein the antibody is characterized by an affinity KD for cynomolgus TNFR2 of less than a figure selected from group consisting of 1 nm and 0.1 nm.

In some aspects, the affinity is measured by Surface Plasmon Resonance (SPR). In some aspects, the SPR was measured using a BIAcore. In some aspects, the affinity was measured by SPR wherein biotinylated TNFR2 is captured on a streptavidin strip surface for 60 seconds at a flow rate of 10 ml/min, and the antibody is injected over the captured TNFR2 at concentrations ranging for an association phase of 50 seconds at 80 ml/min, and the dissociation phase then initiated with running HBS-EP+ buffer injected for 600 seconds at 80 ml/min. In some aspects, the antibody is injected at a concentration ranging from 10 to 0.625 nM. In some aspects, particularly for standard IgG formats comprising two antigen binding domains, the antibody is injected at a concentration ranging from 10 to 1.25 nM. In some aspects, particularly tetrafab formats comprising four antigen binding domains, the antibody is injected at a concentration ranging from 5 to 1.25 nM. IN some aspects, the SPR data is analyzed using Biacore Evaluation Software.

### Polynucleotides Encoding Anti-TNFR2 Antibodies, and Methods of Manufacture

The disclosure also provides polynucleotides encoding any of the antibodies of the invention, including antibody portions and modified antibodies described herein. The invention also provides a method of making any of the antibodies and polynucleotides described herein. Polynucleotides can be made and the proteins expressed by procedures known in the art.

If desired, an anti-TNFR2 antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an anti-TFR2 antibody provided herein. The sequence encoding the antibody of interest may be maintained in a vector in a host cell and the host cell can then be expanded and frozen for future use. Vectors (including expression vectors) and host cells are further described herein.

In some embodiments, the disclosure provides polynucleotides encoding the amino acid sequences of any of the following anti-TNFR2 antibodies IgG2-854, IgG2-1765, and IgG1. In one embodiment, the invention provides polynucleotides encoding the amino acid sequence of anti-TNFR2 antibody TF-2053

In some embodiments, the disclosure provides polynucleotides encoding one or more anti-TNFR2 antibody heavy chain polypeptides comprising an amino acid sequence selected from the group consisting of SEQ ID NO: 19, SEQ ID NO: 22, and SEQ ID NO: 30.

In some embodiments, the disclosure provides polynucleotides encoding one or more anti-TNFR2 antibody light chain polypeptides comprising an amino acid sequence selected from the group consisting of: SEQ ID NO: 6, and SEQ ID NO: 9.

In some embodiments, the disclosure provides polynucleotides encoding one or more anti-TNFR2 antibody VH polypeptides comprising an amino acid sequence selected from the group consisting of: SEQ ID NO: 13, SEQ ID NO: 21, SEQ ID NO: 29.

In some embodiments, the disclosure provides polynucleotides encoding one or more anti-TNFR2 antibody VL polypeptides comprising an amino acid sequence selected from the group consisting of: SEQ ID NO: 4, and SEQ ID NO: 8.

The invention provides a polynucleotide comprising the nucleic acid sequence of the insert of the plasmid deposited with the ATCC and having Accession No. PTA-127530 encoding the heavy chain of antibody Tetrafab 2053. The invention also provides a polynucleotide comprising the nucleic acid sequence of the insert of the plasmid deposited with the ATCC and having Accession No. PTA-127532 encoding the light chain of antibody Tetrafab-2053. In addition, the invention provides a polypeptide comprising the amino acid sequence encoded by the DNA insert of the plasmid deposited with the ATCC and having Accession No. PTA-127528, encoding a VH domain of antibody Tetrafab-2053. The invention further provides a polypeptide comprising the amino acid sequence encoded by the DNA insert of the plasmid deposited with the ATCC and having Accession No. PTA-127529, encoding a VH domain of antibody Tetrafab-2053. The invention further provides a polypeptide comprising the amino acid sequence encoded by the insert of the plasmid deposited with the ATCC and having Accession No. PTA-127531 encoding the VL domain of antibody Tetrafab-2053.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein.

Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

### Pharmaceutical Compositions

In another embodiment, the invention comprises pharmaceutical compositions.

A “pharmaceutical composition” refers to a mixture of an antibody the invention and one or excipient.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

### Therapeutic, Diagnostic, and Other Methods

The antibodies and the antibody conjugates of the present invention are useful in various applications including, but are not limited to, therapeutic treatment methods and diagnostic treatment methods.

In some embodiments, antibodies of the invention may stimulate the activity of TNFR2 and may be useful in the treatment, prevention, suppression and amelioration of disease(s) rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE) or diseases, disorders and conditions by stimulating TNFR2.

In one aspect, the invention provides a method for treating one or more selected from the group consisting of rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE). In some embodiments, the method of treating one or more selected from the group consisting of rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE) in a subject comprises administering to the subject in need thereof an effective amount of a pharmaceutical composition comprising any of the TNFR2 antibodies as described herein. In some embodiments, provided is a method of reducing inflammation and/or activating or expanding immune regulatory cell types in a subject, comprising administering to the subject in need thereof an effective amount of a composition comprising an antibody provided herein.

In another aspect, the invention further provides an antibody or pharmaceutical composition as described herein for use in the described method of treating rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE) or other autoimmune and inflammatory conditions. The invention also provides the use of an antibody as described herein in the manufacture of a medicament for treating one or more selected from the group consisting of rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE) or other autoimmune and inflammatory conditions.

In another aspect, provided is a method of one or more of detecting, diagnosing, or monitoring one or more selected from the group consisting of rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE) or other autoimmune and inflammatory conditions. For example, the anti-TNFR2 antibodies as described herein can be labeled with a detectable moiety such as an imaging agent and an enzyme-substrate label. The antibodies as described herein can also be used for in vivo diagnostic assays, such as in vivo imaging (e.g., PET or SPECT), or a staining reagent.

With respect to all methods described herein, reference to anti-TNFR2 antibodies also includes pharmaceutical compositions comprising the anti-TNFR2 antibodies and one or more additional agents.

### Administration and Dosing

Typically, an antibody of the invention is administered in an amount effective to treat a condition as described herein. The antibodies the invention can be administered as an antibody per se, or alternatively, as a pharmaceutical composition containing the antibody.

The antibodies of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the antibodies of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely. In one embodiment, the total daily dose of an antibody of the invention is typically from about 0.01 to about 100 mg/kg (i.e., mg antibody of the invention per kg body weight) for the treatment of the indicated conditions discussed herein. In another embodiment, total daily dose of the antibody of the invention is from about 0.1 to about 50 mg/kg, and in another embodiment, from about 0.5 to about 30 mg/kg.

### Co-Administration

The antibodies of the invention can be used alone, or in combination with one or more other therapeutic agents. The invention provides any of the uses, methods or compositions as defined herein wherein an antibody of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

### Kits

Another aspect of the invention provides kits comprising the antibody of the invention or pharmaceutical compositions comprising the antibody. A kit may include, in addition to the antibody of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent.

In yet another embodiment, the invention comprises kits that are suitable for use in performing the methods of treatment described herein. In one embodiment, the kit contains a first dosage form comprising one or more of the antibodies of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

### Biological Deposits

Representative materials of the present invention were deposited in the American Type Culture Collection, 10801 University Boulevard, Manassas, VA 20110-2209, USA, on Mar. 3rd, 2023.

| Antibody | Description | ATCC Accession No. |
|---|---|---|
| 2053_outerVH | TF2053 outer VH domain | PTA-127528 |
| 2053_InnerVH | TF2053 inner VH domain | PTA-127529 |
| 2053_FullVH | TF2053 full HC | PTA-127530 |
| 2053_VL | TF2053 VL | PTA-127531 |
| 2053_FullVL | TF2053 full LC | PTA-127532 |

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638). The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws.

### EXAMPLES

The following examples of specific aspects for carrying out the present invention are offered for illustrative purposes only and are not intended to limit the scope of the present invention in any way.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

| Abbreviations | Meaning |
|---|---|
| AUC | Area under the curve |
| CDR | Complementarity determining region |
| CH | Constant heavy |
| CHO | Chinese Hamster Ovary |
| CRD | Cysteine-rich domain |
| Cyno | Cynomolgus monkey |
| DAPI | |
| EC50 | Half maximum effective concentration |
| F | Female |
| Fab | Fragment antigen binding |
| FBS | Fetal bovine serum |
| Fc | Fragment crystallizable |
| FW | Framework |
| G4S | Glycine-glycine-glycine-glycine-serine |
| GFP | Green fluorescent protein |
| ICAM-1 | Intercellular adhesion molecule-1 |
| IKBa | Nuclear factor of kappa light polypeptide |
| | gene enhancer in B-cells inhibitor alpha |
| IL-2 | Interleukin-2 |
| IP | Intra-peritoneal |
| IV | Intravenous |
| M | Male |
| MFI | Mean fluorescence intensity |
| NF-KB | Nuclear factor kappa B |
| OX40 | OX40 receptor, also known as TNFRSF4 |
| | or CD134 |
| PBMC | Peripheral blood mononuclear cells |
| Pen/Strep | Penicillin/streptomycin |
| PFA | Paraformaldehyde |
| PMA | Phorbol 12-myristate 13-acetate |
| SDR | Specificity determining residues |
| SEC | Size exclusion column |
| SPR | Surface Plasmon Resonance |
| TIGIT | T cell immunoreceptor with |
| | immunoglobulin and ITIM domain |
| TNFa | Tumour necrosis factor alpha |
| TNFR2 | Tumour Necrosis Factor Receptor-2 |
| Treg | Regulatory T cells |

### Example 1 Isolation of Mouse Monoclonal Antibodies that Bind to Human and Cynomolgus Monkey TNFR2

SJL mice were immunized with several IP injections of Enbrel (HuTNFR2-IgG1-Fc) with ribi as adjuvant. Sera were screened for binding to human and cynomolgus monkey (Cyno) TNFR2 over-expressed on the surface of CHO cells. Mice showing strong binding on CHO cells were euthanized and isolated B cells were fused with P3X myelomas by electrofusion.

Following hybridoma supernatant screening, using TNFR2 CHO binding, 14 clones were selected and reformatted into HulgG1 and HulgG2 (see example 2). Of these 28 HulgG1 and HulgG2, after confirmatory screening on CHO cells over-expression human and cyno TNFR2 (see table 1), 16 were selected for further characterization (clones 813, 817, 836, 846, 851, 854, 1201, 1070, 1211, 1215, 1296, 1302 1304, 1310, 1327, and 1329), showing a range of binding to human and cyno TNFR2.

TABLE 1
Human and cyno TNFR2 CHO cells EC50 values of
HuIgG1 and HuIgG2 pairs of selected clones.

| | Hu CHO | Cy CHO | | Hu CHO | Cy CHO |
|---|---|---|---|---|---|
| IgG1 | EC50 | EC50 | IgG2 | EC50 | EC50 |
| clone | (ng/ml) | (ng/ml) | clone | (ng/ml) | (ng/ml) |
| 813 | 7557 | nd | 842 | 2379 | nd |
| 807 | 147.1 | 144.4 | 836 | 50.9 | 162 |
| 817 | 89.9 | 105.3 | 846 | 135 | 233 |
| 822 | 96.91 | nd | 851 | 363.1 | nd |
| 825 | 69.8 | nd | 854 | 106.2 | 130 |
| 899 | 35.2 | 149 | 1334 | 85.70 | 210.4 |
| 957 | intermediate | Weak binding | 1201 | 163.2 | 33 |
| | binding | | | | |
| 1070 | 356.0 | 748.1 | 1209 | 2644 | 18500 |
| 1072 | nd | nd | 1211 | 245.0 | nd |
| 1076 | 79.2 | 279.4 | 1215 | 216.3 | 271 |
| 1296 | 160.2 | 412.3 | 1321 | 4250 | 5537 |
| 1304 | 234.0 | Weak binding | 1329 | 163.4 | Strong binding |
| 1285 | nd | nd | 1310 | 151.4 | nd |
| 1302 | 154.6 | 257.4 | 1327 | 337.5 | 486 |

Nd: not determined (some clones were poor expressers and the amount of material to test was limited)

### Example 2 Cloning of Mouse Anti-TNFR2 Antibody Heavy and Light Chain Variable Regions

Heavy chain and light chain variable regions of the anti-TNFR2 antibodies were cloned using the SMART® cDNA synthesis system (Takara Bio Inc. of Shiga, Japan) followed by PCR amplification. The cDNA was synthesized from 1 ug total RNA isolated from approximately 500,000 hybridoma cells using the RNEasy kit (Qiagen) and a template switching oligo with SuperscriptIV™ reverse transcriptase (Invitrogen). The cDNA was then amplified by PCR using a primer that anneals to the SMART® IIA oligo sequence and a mouse constant region-specific primer (mouse kappa for the light chain and mouse IgG for the heavy chain) with Q5 High-Fidelity 2× Master Mix (New England Biolabs). The variable heavy and light chain regions were cloned into mouse pTT5 mammalian expression vector containing the mouse IgG2a constant region and kappa constant regions, respectively, using the infusion cloning method Takara Bio) and the nucleic acid sequences were determined.

The variable heavy regions were then cloned into the pTT5 expression vector containing a human IgG1 constant region mutated to abolish effector function (Leu234Ala, Leu235Ala and Gly237Ala, EU numbering; U.S. Pat. No. 5,624,821) producing chimeric heavy chains. Variable light regions were cloned in the pTT5 mammalian expression vector containing the constant human kappa region to produce chimeric light chains.

### Example 3 Some Anti-TNFR2 Antibodies Agonize Human TNFR2 in NF-KB-GFP Jurkat Cells Reporter Cell Assay

NF-KB-GFP Jurkat cells (System Biosciences, Palo Alto, CA) were stably transfected by transduction using a lentiviral vector containing a full length-human TNFR2 insert (pLVX-puro, Takara Bio US). For the assay, 100 000 cells/well were plated in a 96 well plate, in RPM11640 media supplemented with 10% FBS, 1× sodium pyruvate, 1× Glutamax and Pen/Strep. The cells were next incubated overnight at 37° C. with serial dilutions of anti-TNFR2 antibodies. The following day, the cells were prepared for flow cytometry analysis as follows: after centrifugation to remove the supernatant, the cells were treated with 0.25% trypsin to break up clumps. Finally, the cells were centrifuged again, resuspended in 75 ml of flow buffer containing 1/1000 dilution of DAPI reagent and analyzed on a flow cytometer for GFP expression

Table 2 summarizes the results of the previous TNFR2 CHO cells assay, and the potency in the Jurkat cells reporter assay. We chose 9 clones, all IgG2, to move forward for further characterization, based on a range of EC50 on the Jurkat assay, and sequence diversity.

TABLE 2
CHO binding and Jurkat reporter assay
EC50 of a range of selected clones

| Clone | Isotype | Hu CHO (ng/ml) | Cy CHO (ng/ml) | Jurkat (ng/ml) |
|---|---|---|---|---|
| 813 | IgG1 | 7557 | nd | 1122 |
| 836 | IgG2 | 50.9 | 162 | 598 |
| 846 | IgG2 | 135 | 233 | 142 |
| 817 | IgG1 | 89.9 | 105.3 | 170.1 |
| 851 | IgG2 | 363.1 | nd | 263 |
| 854 | IgG2 | 106.2 | 130 | 711 |
| 1201 | IgG2 | 163.2 | 33 | 11.6 |
| 1070 | IgG1 | 356.0 | 748.1 | 112.9 |
| 1211 | IgG2 | 245.0 | nd | 130 |
| 1215 | IgG2 | 216.3 | 271 | 492 |
| 1296 | IgG1 | 160.2 | 412.3 | 3169 |
| 1304 | IgG1 | 234.0 | poor | Not agonistic |
| 1329 | IgG2 | 163.4 | Strong binding | 60 |
| 1310 | IgG2 | 151.4 | nd | 667 |
| 1302 | IgG1 | 154.6 | 257.4 | 660.9 |
| 1327 | IgG2 | 337.5 | 486 | Not agonistic |

Nd: not determined (some clones were poor expressers and the amount of material to test was limited)

### Example 4 Biophysical Characteristics and Agonism in Primary Cells (Human PBMC Activation Assay) of Selected Agonist Clones

The 9 previously selected clones were next tested for their ability to induce ICAM-1 up-regulation in CD3+CD4+TIGIT+ human PBMC (see example 11 for method). We also assessed some biophysical properties such as sequence liabilities and non-specificity (AC-SINS and polyreactivity).

TABLE 3
sequence liabilities, non-specificity levels
and agonism in human primary cells.

| | Sequence | | polyreac- | Agonism in Human |
|---|---|---|---|---|
| Clone | liabilities | AC-SINS | tivity | PBMC |
| 836 | N-linked | Low | Low | Intermediate agonist |
| | glycosylation | | | |
| | site | | | |
| 846 | Oxidation sites | Low | High | Intermediate agonist |
| 851 | Isomerization | Intermediate | High | Weak agonist |
| | and | | | |
| | deamidation | | | |
| | sites | | | |
| 854 | Oxidation and | Intermediate | Low | strong agonist |
| | deamidation | | | |
| | site | | | |
| 1201 | Oxidation sites | Low | Low | Weak agonist |
| 1211 | Isomerization | Low | High | Weak agonist |
| | and | | | |
| | deamidation | | | |
| | sites | | | |
| 1215 | Free cysteine | Low | High | Weak agonist |
| | in CDR | | | |
| | Oxidation sites | | | |
| 1310 | Oxidation site | Intermediate | Low | Medium agonist |
| 1329 | N-linked | High | High | Not agonist |
| | glycosylation | | | |
| | site | | | |

As seen in table 3, we observed a range of agonism on human PBMC, and decided to move forward the clones showing some potency on primary cells (clones 836, 846, 854, 1201 and 1310) and with non-specificity levels we could manage by optimisation later if necessary.

### Example 6 Epitope Binning of Anti-TNFR2 Antibodies

Epitope binning using yeast expressing human TN FR1/TNFR2 domains chimera was also performed. Yeast cells were stably transfected with various constructs, where one or two CRD of TNFR2 was swapped for the corresponding human TNFR1 CRD. Similarly, the reverse constructs were also tested, where one or two CRD of TNFR1 were swapped for the corresponding TNFR2 CRD. Binding loss or gain, respectively, indicated that particular CRD was necessary for binding to TNFR2 and contained the epitope.

As seen in table 4, most clones bind to CRD3-4, except for clone 836 (CRD1) and clone 1310 (CRD1-3-4).

TABLE 4
The binding to human TNFR1/TNFR2 chimera expressed on yeast
was assessed to determine the binding domain on TNFR2

| clone | epitope |
|---|---|
| 836 | CRD1 |
| 846 | CRD3-4 |
| 854 | CRD3-4 |
| 1201 | CRD-3-4 |
| 1310 | CRD1-3-4 |
| 1329 | CRD3-4 |

### Example 7 Humanization of Mouse Anti-TNFR2 Antibodies

Clone 854 was chosen for further humanization since it was the stronger agonistic antibody. Humanization of clone 854 was performed by grafting the murine hypervariable regions into various human frameworks.

The hypervariable regions of the heavy chain were defined using the SDR definition (Specificity Determining Residues), whereas the hypervariable regions of the light chain were defined using the CDR definition (Complementarity Determining Region).

TABLE 5
amino acid sequence of the heavy and light chains of clone 854.

| | FW1 | SDR1 | FW2 | SDR2 | WF3 | SDR3 | FW4 |
|---|---|---|---|---|---|---|---|
| Heavy | Residues | GFSFPSSGV | Residues | VTWGVGS | Residues | GEW | Residues |
| chain | 1-25 of | D | 36-49 | THYNSAL | 66-97 of | DEGFVYW | 107- 117 of |
| (SEQ ID | SEQ ID | (SEQ ID NO: | of SEQ ID | KS | SEQ ID | GQG | SEQ ID |
| NO: 13) | NO: 13 | 10) | NO: 13 | (SEQ ID | NO: 13 | (SEQ ID | NO: 13 |
| | | | | NO: 11) | | NO: 12) | |
| | FW1 | CDR1 | FW2 | CDR2 | WF3 | CDR3 | FW4 |
| Light | Residues | KASQNVGTA | Residues | SASNRYT | Residues | QQYSSYP | Residues |
| chain | 1-23 of | VA | 35-49 of | (SEQ ID | 57-88 of | LT | 97-107 of |
| (SEQ ID | SEQ ID | (SEQ ID NO: | SEQ ID | NO: 2) | SEQ ID | (SEQ ID | SEQ ID |
| NO: 4) | NO: 4 | 1) | NO: 4 | | NO: 4 | NO: 3) | NO: 4 |

The SDR definition is used for grafting, rather than the CDR, so murine amino acids critical for binding and affinity are conserved during the humanization process.

The human heavy chain frameworks chosen for grafting of the 854 murine heavy chain SDRs were the following: IGHV4-31*02, IGHV4-30-4*07, IGHV4-4*08 (the 3 closest germline families to clone 854), IGHV3-23*01, IGHV1-46*01, and IGHV3-7*01 (3 commonly used human frameworks), in conjunction with the IGHJ4*01 J-gene. The human light chain frameworks chosen for grafting of the 854 murine light chain CDRs were the following: IGKV3-11*01, IGKV1-9*01, IGKV1-33*01, IGKV1-27*01, IGKV1-39*01, and IGKV4-1*01, in conjunction with the IGKJ4*01 J-gene.

### Example 8 Binding to Human TNFR2 CHO Cells by Humanized TNFR2 Antibodies

All combination of heavy and light chains were expressed and tested for binding to Human TNFR2 CHO cells:

TABLE 6
Human grafts binding data - combinations of heavy and light chains were purified
and tested for binding to human TNFR2 CHO cells. Results are expressed as EC50
and % of binding compared to the maximum signal of the parental 854.

| | | | | % max | |
|---|---|---|---|---|---|
| | | | | signal to | |
| VH | VK | name | EC50 nM | 854 | AC-SINS |
| | | GBT-TNFR2-854 (parental) | 1.131 | 100 | |
| IGHV4-31 | IGKV1-9 | GBT-TNFR2-0854_Graft1 | 31.71 | 48 | Moderate |
| IGHV4-31 | IGKV1-33 | GBT-TNFR2-0854_Graft2 | 43.77 | 34 | Moderate |
| IGHV4-31 | IGKV1-27 | GBT-TNFR2-0854_Graft3 | 90.01 | 22 | Moderate |
| IGHV4-31 | IGKV1-39 | GBT-TNFR2-0854_Graft4 | 38.23 | 31 | Moderate |
| IGHV4-31 | IGKV3-11 | GBT-TNFR2-0854_Graft5 | 42.95 | 41 | Low |
| IGHV4-31 | IGKV4-1 | GBT-TNFR2-0854_Graft6 | 65.38 | 27 | Low |
| IGHV4-30-4 | IGKV1-9 | GBT-TNFR2-0854_Graft7 | 271.2 | 51 | Moderate |
| IGHV4-30-4 | IGKV1-33 | GBT-TNFR2-0854_Graft8 | 87.91 | 29 | Moderate |
| IGHV4-30-4 | IGKV1-27 | GBT-TNFR2-0854_Graft9 | 125.2 | 22 | Moderate |
| IGHV4-30-4 | IGKV1-39 | GBT-TNFR2-0854_Graft10 | 53.69 | 33 | Moderate |
| IGHV4-30-4 | IGKV3-11 | GBT-TNFR2-0854_Graft11 | 41.04 | 34 | Low |
| IGHV4-30-4 | IGKV4-1 | GBT-TNFR2-0854_Graft12 | 50.96 | 27 | Low |
| IGHV4-4 | IGKV1-9 | GBT-TNFR2-0854_Graft13 | 28.44 | 29 | Moderate |
| IGHV4-4 | IGKV1-33 | GBT-TNFR2-0854_Graft14 | 120.6 | 24 | Moderate |
| IGHV4-4 | IGKV1-27 | GBT-TNFR2-0854_Graft15 | 143.3 | 15 | Moderate |
| IGHV4-4 | IGKV1-39 | GBT-TNFR2-0854_Graft16 | 42.91 | 34 | Moderate |
| IGHV4-4 | IGKV3-11 | GBT-TNFR2-0854_Graft17 | 47.82 | 36 | Low |
| IGHV4-4 | IGKV4-1 | GBT-TNFR2-0854_Graft18 | 59.48 | 28 | Low |
| IGHV3-23 | IGKV1-9 | GBT-TNFR2-0854_Graft19 | 9.437 | 56 | Low |
| IGHV3-23 | IGKV1-33 | GBT-TNFR2-0854_Graft20 | 13.75 | 52 | Moderate |
| IGHV3-23 | IGKV1-27 | GBT-TNFR2-0854_Graft21 | 27.55 | 50 | Low |
| IGHV3-23 | IGKV1-39 | GBT-TNFR2-0854_Graft22 | 17.99 | 58 | Low |
| IGHV3-23 | IGKV3-11 | GBT-TNFR2-0854_Graft23 | 26.35 | 51 | Low |
| IGHV3-23 | IGKV4-1 | GBT-TNFR2-0854_Graft24 | 27.99 | 47 | Low |
| IGHV3-7 | IGKV1-9 | GBT-TNFR2-0854_Graft25 | 10.96 | 56 | Low |
| IGHV3-7 | IGKV1-33 | GBT-TNFR2-0854_Graft26 | 15.58 | 45 | Low |
| IGHV3-7 | IGKV1-27 | GBT-TNFR2-0854_Graft27 | 13.48 | 64 | Low |
| IGHV3-7 | IGKV1-39 | GBT-TNFR2-0854_Graft28 | 19.52 | 62 | Low |
| IGHV3-7 | IGKV3-11 | GBT-TNFR2-0854_Graft29 | 26.07 | 46 | Low |
| IGHV3-7 | IGKV4-1 | GBT-TNFR2-0854_Graft30 | 15.92 | 55 | Low |
| IGHV1-46 | IGKV1-9 | GBT-TNFR2-0854_Graft31 | 1.873 | 79 | High |
| IGHV1-46 | IGKV1-33 | GBT-TNFR2-0854_Graft32 | 4.995 | 74 | High |
| IGHV1-46 | IGKV1-27 | GBT-TNFR2-0854_Graft33 | 2.97 | 72 | High |
| IGHV1-46 | IGKV1-39 | GBT-TNFR2-0854_Graft34 | 2.579 | 78 | High |
| IGHV1-46 | IGKV3-11 | GBT-TNFR2-0854_Graft35 | 1.579 | 78 | High |
| IGHV1-46 | IGKV4-1 | GBT-TNFR2-0854_Graft36 | 5.436 | 66 | Moderate |

After grafting into various human frameworks, a significant loss of binding was observed, as summarized in table 6. Some clones, such as grafts 31, 33, 34 and 35, displayed an EC50 value close to 854 but the maximum signal was significantly lower, and their AC-SINS score were high. All DNA/insulin polyreactivity scores were low (data not shown).

### Example 9 Introduction of Back-Mutations to Restore Binding to TNFR2

To restore binding to TNFR2 to the level of the parental 854 clone, various back-mutations were introduced into a selected number of heavy chains grafted constructs:

TABLE 7
back-mutations introduced into the human heavy
chain frameworks VH1-46, VH3-7, and VH4-30.

| FW | Introduced mutations (Kabat numbering) |
|---|---|
| VH1-46 | A24V |
| VH1-46 | M69I, R71K |
| VH1-46 | A24V, M69I, R71K |
| VH1-46 | A24V, M69I, R71K, T75K |
| VH1-46 | A24V, M69I, R71K, T73N, T75K |
| VH1-46 | A24V, Q61S, M69I, R71K |
| VH3-7 | L78V |
| VH3-7 | R71K, L78V |
| VH3-7 | A24V, L78V |
| VH3-7 | A24V, R71K |
| VH4-30 | V71K |
| VH4-30 | F78V |
| VH4-30 | V71K, F78V |
| VH4-30 | V71K, T73N, F78V |
| VH4-30 | V71K, N76S, F78V |
| VH4-30 | V71K, F78V, V89I |
| VH4-30 | I37V, V71K, F78V |

Each heavy chain was paired with the following humanized light chain: VK4-1, VK3-11, VK1-39 and VK1-9 as described in example 7, but also with the same humanized light chain where the CDR2-L2 is germlined.

### Example 10 Binding to Human TNFR2 by Humanized TNFR2 Antibodies

A total of 114 clones were expressed and purified, and their binding to Human TNFR2 CHO cells was compared to the parental 854 clone. As seen in table 8 below, most of the clones displayed EC50 close to or better than the parental clone 854. Clones, 1847, 1765, 1753 and 1747 were selected for further characterization.

TABLE 8
Binding and AC-SINS data of the humanized clones carrying back-mutations.

| clone | | | CHO EC50 | AC- |
|---|---|---|---|---|
| names | Heavy Chain | Light chain | nM | SINS |
| Parental | | | 1.131 | |
| 854 | | | | |
| 1848 | VH4-30_I37V_V71K_F78V | VK1-9 parental | 2.541 | Low |
| 1847 | VH4-30_I37V_V71K_F78V | VK1-9_GL CDR2 | 1.309 | Low |
| 1846 | VH4-30_I37V_V71K_F78V | VK3-11 parental | 1.851 | Low |
| 1845 | VH4-30_I37V_V71K_F78V | VK3-11_GL CDR2 | 1.277 | Low |
| 1844 | VH4-30_I37V_V71K_F78V | VK1-39 parental | 2.471 | Low |
| 1843 | VH4-30_I37V_V71K_F78V | VK1-39_GL CDR2 | 4.407 | Low |
| 1842 | VH4-30_V71K_F78V_V89I | VK1-9 parental | 3.543 | Low |
| 1841 | VH4-30_V71K_F78V_V89I | VK1-9_GL CDR2 | 3.311 | Low |
| 1840 | VH4-30_V71K_F78V_V89I | VK3-11 parental | 1.638 | Low |
| 1839 | VH4-30_V71K_F78V_V89I | VK3-11_GL CDR2 | 1.547 | Low |
| 1838 | VH4-30_V71K_F78V_V89I | VK1-39 parental | 1.886 | Low |
| 1837 | VH4-30_V71K_F78V_V89I | VK1-39_GL CDR2 | 3.024 | Low |
| 1836 | VH4-30_V71K_N76S_F78V | VK1-9 parental | 2.968 | Low |
| 1835 | VH4-30_V71K_N76S_F78V | VK1-9_GL CDR2 | 2.988 | Low |
| 1834 | VH4-30_V71K_N76S_F78V | VK3-11 parental | 2.931 | Low |
| 1833 | VH4-30_V71K_N76S_F78V | VK3-11_GL CDR2 | 2.263 | Low |
| 1832 | VH4-30_V71K_N76S_F78V | VK1-39 parental | 2.113 | Low |
| 1831 | VH4-30_V71K_N76S_F78V | VK1-39_GL CDR2 | 1.888 | Low |
| 1830 | VH4-30_V71K_T73N_F78V | VK1-9 parental | 2.851 | Low |
| 1829 | VH4-30_V71K_T73N_F78V | VK1-9_GL CDR2 | 2.53 | Low |
| 1828 | VH4-30_V71K_T73N_F78V | VK3-11 parental | 2.769 | Low |
| 1827 | VH4-30_V71K_T73N_F78V | VK3-11_GL CDR2 | 2.403 | Low |
| 1826 | VH4-30_V71K_T73N_F78V | VK1-39 parental | 3.853 | Low |
| 1825 | VH4-30_V71K_T73N_F78V | VK1-39_GL CDR2 | 4.494 | Low |
| 1824 | VH4-30_V71K_F78V | VK1-9 parental | 1.612 | Low |
| 1823 | VH4-30_V71K_F78V | VK1-9_GL CDR2 | 2.149 | Low |
| 1822 | VH4-30_V71K_F78V | VK3-11 parental | 1.942 | Low |
| 1821 | VH4-30_V71K_F78V | VK3-11_GL CDR2 | 1.64 | Low |
| 1820 | VH4-30_V71K_F78V | VK1-39 parental | 2.458 | Low |
| 1819 | VH4-30_V71K_F78V | VK1-39_GL CDR2 | 3.228 | Low |
| 1818 | VH4-30_F78V | VK1-9 parental | 350.7 | Low |
| 1817 | VH4-30_F78V | VK1-9_GL CDR2 | 771 | Low |
| 1816 | VH4-30_F78V | VK3-11 parental | 617.8 | Low |
| 1815 | VH4-30_F78V | VK3-11_GL CDR2 | 215.1 | Low |
| 1814 | VH4-30_F78V | VK1-39 parental | no binding | Low |
| 1813 | VH4-30_F78V | VK1-39_GL CDR2 | 35.59 | Low |
| 1812 | VH4-30_V71K | VK1-9 parental | 8.323 | High |
| 1811 | VH4-30_V71K | VK1-9_GL CDR2 | 9.518 | Low |
| 1810 | VH4-30_V71K | VK3-11 parental | 17.83 | Low |
| 1809 | VH4-30_V71K | VK3-11_GL CDR2 | 7.181 | Low |
| 1808 | VH4-30_V71K | VK1-39 parental | 24.48 | High |
| 1807 | VH4-30_V71K | VK1-39_GL CDR2 | 181.1 | High |
| 1806 | VH3-7_A24V_R71K | VK1-9 parental | 12.07 | Low |
| 1805 | VH3-7_A24V_R71K | VK1-9_GL CDR2 | 1.173 | Low |
| 1804 | VH3-7_A24V_R71K | VK3-11 parental | 55.61 | Low |
| 1803 | VH3-7_A24V_R71K | VK3-11_GL CDR2 | 12.59 | Low |
| 1802 | VH3-7_A24V_R71K | VK1-39 parental | 19.81 | Low |
| 1801 | VH3-7_A24V_R71K | VK1-39_GL CDR2 | 33.97 | Low |
| 1800 | VH3-7_A24V_L78V | VK1-9 parental | 1.151 | Low |
| 1799 | VH3-7_A24V_L78V | VK1-9_GL CDR2 | 2.001 | Low |
| 1798 | VH3-7_A24V_L78V | VK3-11 parental | 0.5211 | Low |
| 1797 | VH3-7_A24V_L78V | VK3-11_GL CDR2 | 0.6889 | Low |
| 1796 | VH3-7_A24V_L78V | VK1-39 parental | 4.543 | Low |
| 1795 | VH3-7_A24V_L78V | VK1-39_GL CDR2 | 3.963 | Low |
| 1794 | VH3-7_R71K_L78V | VK1-9 parental | 11.41 | Low |
| 1793 | VH3-7_R71K_L78V | VK1-9_GL CDR2 | 13.47 | Low |
| 1792 | VH3-7_R71K_L78V | VK3-11 parental | 14.71 | Low |
| 1791 | VH3-7_R71K_L78V | VK3-11_GL CDR2 | 19.12 | Low |
| 1790 | VH3-7_R71K_L78V | VK1-39 parental | 14.64 | Low |
| 1789 | VH3-7_R71K_L78V | VK1-39_GL CDR2 | 24.37 | Low |
| 1788 | VH3-7_L78V | VK1-9 parental | 36.45 | Low |
| 1787 | VH3-7_L78V | VK1-9_GL CDR2 | 32.53 | Low |
| 1786 | VH3-7_L78V | VK3-11 parental | 87.75 | Low |
| 1785 | VH3-7_L78V | VK3-11_GL CDR2 | 80.24 | Low |
| 1784 | VH3-7_L78V | VK1-39 parental | 76.35 | Low |
| 1783 | VH3-7_L78V | VK1-39_GL CDR2 | 96.87 | Low |
| 1782 | VH1-46_A24V_Q61S_M69I_R71K | VK1-9 parental | 1.55 | Low |
| 1781 | VH1-46_A24V_Q61S_M69I_R71K | VK1-9_GL CDR2 | 1.618 | Low |
| 1780 | VH1-46_A24V_Q61S_M69I_R71K | VK3-11 parental | 1.713 | Low |
| 1779 | VH1-46_A24V_Q61S_M69I_R71K | VK3-11_GL CDR2 | 1.428 | Low |
| 1778 | VH1-46_A24V_Q61S_M69I_R71K | VK1-39 parental | 1.292 | Low |
| 1777 | VH1-46_A24V_Q61S_M69I_R71K | VK1-39_GL CDR2 | 1.588 | Low |
| 1776 | VH1-46_A24V_Q61S_M69I_R71K | VK4-1 parental | 1.476 | Low |
| 1775 | VH1-46_A24V_Q61S_M69I_R71K | VK4-1_GL CDR2 | 2.27 | Low |
| 1774 | VH1-46_A24V_M69I_R71K_T73N_T75K | VK1-9 parental | 1.559 | High |
| 1773 | VH1-46_A24V_M69I_R71K_T73N_T75K | VK1-9_GL CDR2 | 1.13 | High |
| 1772 | VH1-46_A24V_M69I_R71K_T73N_T75K | VK3-11 parental | 1.398 | High |
| 1771 | VH1-46_A24V_M69I_R71K_T73N_T75K | VK3-11_GL CDR2 | 1.456 | High |
| 1770 | VH1-46_A24V_M69I_R71K_T73N_T75K | VK1-39 parental | 1.535 | High |
| 1769 | VH1-46_A24V_M69I_R71K_T73N_T75K | VK1-39_GL CDR2 | 0.9681 | High |
| 1768 | VH1-46_A24V_M69I_R71K_T73N_T75K | VK4-1 parental | 1.573 | Low |
| 1767 | VH1-46_A24V_M69I_R71K_T73N_T75K | VK4-1_GL CDR2 | 1.233 | Low |
| 1766 | VH1-46_A24V_M69I_R71K_T75K | VK1-9 parental | 1.193 | High |
| 1765 | VH1-46_A24V_M69I_R71K_T75K | VK1-9_GL CDR2 | 1.205 | Low |
| 1764 | VH1-46_A24V_M69I_R71K_T75K | VK3-11 parental | 1.002 | Low |
| 1763 | VH1-46_A24V_M69I_R71K_T75K | VK3-11_GL CDR2 | 1.317 | High |
| 1762 | VH1-46_A24V_M69I_R71K_T75K | VK1-39 parental | 1.683 | High |
| 1761 | VH1-46_A24V_M69I_R71K_T75K | VK1-39_GL CDR2 | 1.372 | High |
| 1760 | VH1-46_A24V_M69I_R71K_T75K | VK4-1 parental | 1.586 | Low |
| 1759 | VH1-46_A24V_M69I_R71K_T75K | VK4-1_GL CDR2 | 2.725 | Low |
| 1758 | VH1-46_A24V_M69I_R71K | VK1-9 parental | 1.584 | High |
| 1757 | VH1-46_A24V_M69I_R71K | VK1-9_GL CDR2 | 1.307 | Low |
| 1756 | VH1-46_A24V_M69I_R71K | VK3-11 parental | 1.037 | High |
| 1755 | VH1-46_A24V_M69I_R71K | VK3-11_GL CDR2 | 2.026 | Low |
| 1754 | VH1-46_A24V_M69I_R71K | VK1-39 parental | 1.389 | Low |
| 1753 | VH1-46_A24V_M69I_R71K | VK1-39_GL CDR2 | 0.9743 | Low |
| 1752 | VH1-46_A24V_M69I_R71K | VK4-1 parental | 1.41 | Low |
| 1751 | VH1-46_A24V_M69I_R71K | VK4-1_GL CDR2 | 1.649 | Low |
| 1750 | VH1-46_M69I_R71K | VK1-9 parental | 2.283 | High |
| 1749 | VH1-46_M69I_R71K | VK1-9_GL CDR2 | 1.54 | Low |
| 1748 | VH1-46_M69I_R71K | VK3-11 parental | 2.271 | Low |
| 1747 | VH1-46_M69I_R71K | VK3-11_GL CDR2 | 1.263 | Low |
| 1746 | VH1-46_M69I_R71K | VK1-39 parental | 1.96 | Low |
| 1745 | VH1-46_M69I_R71K | VK1-39_GL CDR2 | 1.759 | Low |
| 1744 | VH1-46_M69I_R71K | VK4-1 parental | 2.291 | Low |
| 1743 | VH1-46_M69I_R71K | VK4-1_GL CDR2 | 3.014 | Low |
| 1742 | VH1-46_A24V | VK1-9 parental | 1.883 | Low |
| 1741 | VH1-46_A24V | VK1-9_GL CDR2 | 1.592 | Low |
| 1740 | VH1-46_A24V | VK3-11 parental | 1.108 | High |
| 1739 | VH1-46_A24V | VK3-11_GL CDR2 | 1.818 | Low |
| 1738 | VH1-46_A24V | VK1-39 parental | 0.8624 | Low |
| 1737 | VH1-46_A24V | VK1-39_GL CDR2 | 1.634 | Low |
| 1736 | VH1-46_A24V | VK4-1 parental | 0.7444 | Low |
| 1735 | VH1-46_A24V | VK4-1_GL CDR2 | 1.731 | Low |

### Example 11 the Humanized Variants Display a Range of Potency in the Human and Cyno PBMC Assays

The selected humanized clones were first tested on the Human TNFR2 Jurkat reporter cell assay. Results in table 9 shows that clone 1765 displays a similar EC50 than the parental clone 854. Both clones 1847 and 1753 lost potency and interestingly, despite being the strongest binder in the Human TNFR2 CHO binding assay, clone 1747 is not an agonist on the Jurkat reporter cell assay.

Following this result, clones 1847, 1765 and 1753 were tested in the human and cyno PBMC assays.

Briefly, 250 000 Human PBMC from various donors were plated in the wells of a 96-well plate in Optimizer CST medium. Each well was treated with 50 ml of 5 ng/ml IL-2 and 50 ml of 4× anti-TNFR2 antibody or TetraFab for 72 hours at 37° C. Subsequently, the cells were prepared for Flow cytometry analysis. After 2 washes in PBS, the cells were first resuspended in 100 ml of Live Near IR staining buffer for 30 minutes at 4° C. After 2 additional washes in flow buffer, the cells were incubated with CD3 BV421, ICAM APC CD4 PE and TIGIT BUV395 diluted 1/40 1 hour at 4° C., then fixed with 1% PFA 15 minutes at room temperature. Following 2 additional washes in flow buffer, the cells were finally analyzed by flow cytometry. Light scatter was used to exclude doublets, and low Near IR staining as an indication of viability. Single, viable cells were gated on the CD3+/CD4+/TIGIT+ population and ICAM-1 mean fluorescence intensity (MFI) measured for ICAM-1. A 4-parameter curve fit in Graph Pad Prism was used to calculate the EC50 of TNFR2 agonist for ICAM-1 induction. ICAM-1 was selected because it is known to be regulated by NF-KB, a marker of acute T cell activation, and has a known role in Treg-mediated suppression. TIGIT was used to identify a population of mature T cells enriched for TNFR2 expression and Treg without having to stain for the intracellular marker FoxP3. A similar strategy was used to determine activation of T cells in cyno PBMC, although T cell activation was measured as the percent double positive CD4+ cells for CD25+/Ki67+ because of differences in activation marker expression and antibody cross reactivity between human and cyno.

The results, in table 9, confirmed that humanized clone 1765 display similar potency as the parental clone 854 in the human PBMC assay, with EC50 of 1.308 and 1.402 nM respectively. The potency on cyno cells was 10-fold lower for both clones, with EC50 of 13.63 and 15.02 nM respectively. Clone 1753 exhibited lower potency, especially on cyno PBMC cells (56 nM) and clone 1847 didn't display any potency on both cell types. Clone 1765 (heavy chain sequence SEQ 22 and light chain sequence SEC 21) was selected for further investigation.

TABLE 9
potency of the humanized clone was tested in the Jurkat reporter
cell assay and in the human and cyno PBMC assays.

| | Jurkat | EC50 | EC50 |
|---|---|---|---|
| | EC50 | (HuPBMC) | (CyPBMC) |
| Clone name | ug/ml | nM | nM |
| GBT-TNFR2-0854 | 1.74 | 1.402 | 15.02 |
| GBT-TNFR2-1847 | 5.97 | Not agonist | Not agonist |
| GBT-TNFR2-1765 | 1.01 | 1.308 | 13.63 |
| GBT-TNFR2-1753 | 13.5 | 4.845 | 56 |
| GBT-TNFR2-1747 | Not agonist | Not tested | Not tested |

### Example 12 Aggregated Anti-TNFR2 Antibodies Display Increased Potency in a Jurkat NF-KB-GFP Reporter Cell Assay

To assess the effect of aggregates on TNFR2 agonism, we compared anti-TNFR2 antibodies purified with a 1-step method (ProA purification only, containing on average 15-20% aggregates) with the same antibodies purified with a 2-steps method (ProA followed by SEC purification, aggregate-free), in a Jurkat NFKB-GFP reporter cell assay.

Briefly, 100 000 NF-KB-GFP Jurkat cells/well were plated in a 96 well plate, in RPM11640 media supplemented with 10% FBS, 1× sodium pyruvate, 1× Glutamax and Pen/Strep. The cells were next incubated overnight at 37° C. with serial dilutions of anti-TNFR2 antibodies. The following day, the cells were prepared for flow cytometry analysis as follows: after centrifugation to remove the supernatant, the cells were treated with 0.25% trypsin to break up clumps. Finally, the cells were centrifuged again, resuspended in 75 ml of flow buffer containing 1/1000 dilution of DAPI reagent and analyzed on a flow cytometer for GFP expression.

TABLE 10
Jurkat reporter cell assay potency of
clones with or without aggregates.

| | EC50 Jurkat assay | EC50 Jurkat assay | |
|---|---|---|---|
| | ng/ml | ng/ml | Loss of |
| Clone | With aggregates | Without aggregates | potency |
| MR2-1 | 119.6 | 5748 | 50-fold |
| Clone 162 | 347.3 | 7021 | 20-fold |

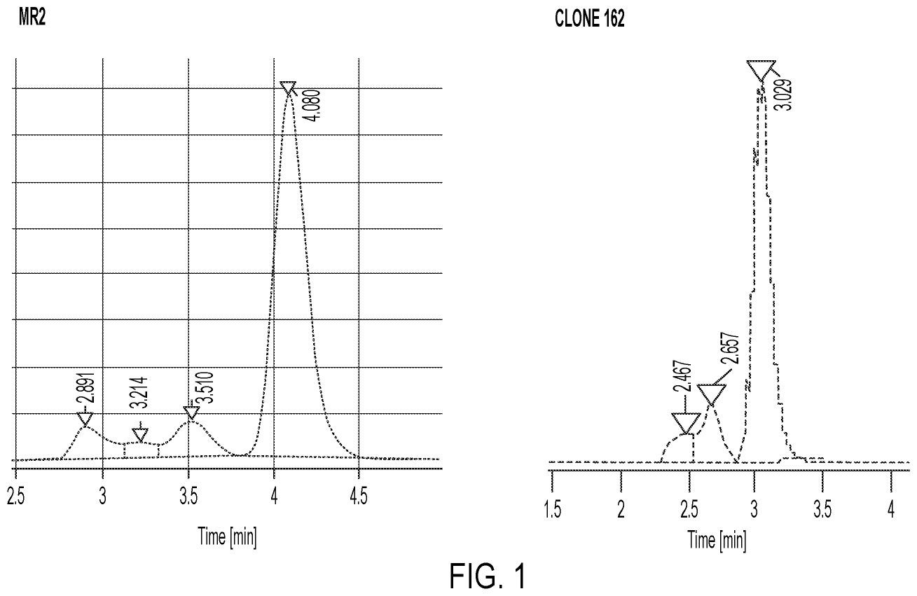

Clone 162, chosen as a representative result, was compared to MR2-1 (Hycult Biotech, ref #HM2007), a commercially available TNFR2 agonist.
When purified on a ProA column, clone 162 displayed a similar potency as MR2-1 (EC50 respectively 347.3 and 119.6 ng/ml). Interestingly, we found out that both clone 162 and MR2-1 contained a significant amount of aggregates, as seen below on the analytical SEC profiles in FIG. 1.

When both clone 162 and MR2-1 were devoid of aggregates, after purification on a SEC, their ability to agonize TNFR2 in the Jurkat cell assay was significantly compromised, with clone 162 showing a 20-fold loss of potency (EC50=7.02 mg/ml) and MR2-1 a 50-fold loss (EC50=5.75 mg/ml), suggesting that the aggregates could artificially increase anti-TNFR2 antibodies potency.

This result prompted us to investigate if increasing valency could lead to increased potency.

### Example 13 Reformatting to TetraFab

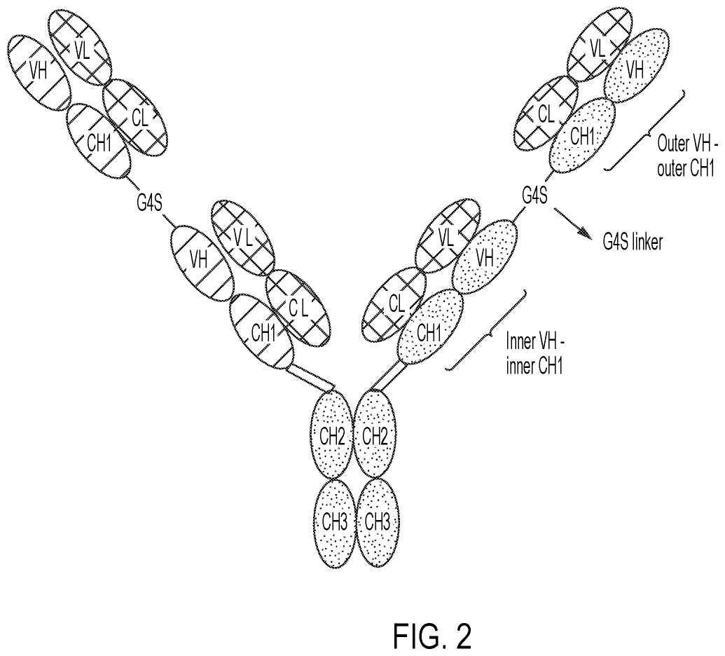

To test our hypothesis, we sought to increase the valency of clone 1765 by adding additional paratopes onto its existing IgG scaffold, reformatting it to TetraFab 2053 (see FIG. 2). As described in the schematic, a TetraFab is comprised of a dual Fab heavy chain, with an outer Fab domain made of a variable domain and a constant CH1 domain linked by a short G4S linker to an inner Fab made of the same variable domain followed by the same constant CH1 and the CH2 and CH3 of a typical human IgG1. This longer heavy chain is able to associate with 4 light chains instead of 2, creating a molecule comprised of 4 paratopes and in theory able to bind 4 molecules of TNFR2. See SEQ 30 and SEQ 9 for TetraFab 2053 heavy and light chains respectively.

The rationale behind the reformatting to TetraFab is to mimic the TNFR2 oligomerisation by membrane-bound TN Fa. Indeed, membrane-bound TNFα forms trimers that able to strongly agonize TNFR2 on the surface of Tregs, by binding multiple TNFR2 molecules simultaneously.

Increased avidity was confirmed by Surface Plasmon Resonance (SPR) using a BIAcore. Briefly, biotinylated human- or cyno-TNFR2 was captured on the surface of a streptavidin chip for 60 seconds at a flow rate of 10 ml/min. Next, the antibodies or the TetraFab were injected over the captured TNFR2 at concentrations ranging from 10 to 1.25 nM for the antibodies, and 5 to 0.625 nM for the TetraFab, for an association phase of 50 seconds at 80 ml/min. The dissociation phase was then initiated with running buffer (HBS-EP+) injected for 600 seconds at 80 ml/min. Data were analyzed using the Biacore Evaluation Software.

TABLE 11
SRP data - On-rate (ka), off-rate (kd) and affinity
(KD) of the parental clone 854, its humanized counterpart
clone 1765 and the corresponding TetraFab 2053 on human
and cyno TNFR2, calculated using a BIACore.

| Analyte | ligand | ka (1/Ms) | kd (1/s) | KD (nM) |
|---|---|---|---|---|
| IgG2 - 854 | HuTNFR2 | 1.13E+07 | 1.25E−03 | 0.11 |
| IgG2 - 1765 | HuTNFR2 | 4.20E+06 | 1.36E−03 | 0.32 ± 0.1 |
| IgG1 TF-2053 | HuTNFR2 | 8.10E+06 | 4.73E−04 | 0.06 ± 0.00 |
| IgG2 - 854 | CyTNFR2 | 1.12E+07 | 9.96E−03 | 0.89 |
| IgG2 - 1765 | CyTNFR2 | 4.09E+06 | 9.77E−03 | 2.39 ± 0.03 |
| IgG1 TF-2053 | CyTNFR2 | 1.15E+07 | 7.06E−03 | 0.61 |

The data in table 11 show that both the parental 854 clone and the humanized 1765 clone have a similar apparent affinity for human TNFR2, with KDs of respectively 0.11 and 0.32 nM. The TetraFab displays a stronger apparent affinity with a KD of 0.06 nM, a 5-fold improvement compared to its IgG counterparts 854 or 1765.

A similar improvement in affinity, albeit not as great, can be observed for cyno TNFR2. The affinity of the TetraFab for cyno TNFR2 is 10-fold less than for human TNFR2 (KDs of 0.61 and 0.06 nM respectively).

### Example 14 Anti-TNFR2 Antibodies and TetraFab Agonize TNFR2 in a Jurkat NF-KB-GFP Reporter Cell Assay

The TetraFab was first tested for its capacity to induce NF-KB-GFP expression in a TNFR2-expressing Jurkat cell reporter assay used previously. Similarly, 100 000 cells/well were plated in a 96 well plate, in RPM11640 media supplemented with 10% FBS, 1× sodium pyruvate, 1× Glutamax and Pen/Strepand incubated overnight at 37° C. with serial dilutions of anti-TNFR2 antibody or TetraFab. The following day, the cells were prepared for flow cytometry analysis as previously and analyzed on a flow cytometer for GFP expression.

The results summarized in table 12 show that the parental clone 854 and its humanized variant 1765 have a similar EC50 of 0.526 mg/ml and 1.009 mg/ml respectively. On the other hand, the EC50 of the TetraFab 2053 shows more than a 10-fold improvement compared to both 854 and 1765, with an EC50 of 0.037 mg/ml. This result suggests that increased valency promotes stronger potency.

TABLE 12
Potency of the parental 854, its humanized counterpart
1765 and the corresponding TetraFab 2053 in
the TNFR2 Jurkat reporter cell assay.

| Clone | EC50 Jurkat μg/ml |
|---|---|
| 854 | 0.526 |
| 1765 | 1.009 |
| 2053 | 0.037 |

### Example 15 Anti-TNFR2 Antibodies and TetraFab Induce ICAM-1 Up-Regulation in CD3+CD4+TIGIT+ Human PBMC and Ki67/CD25 Co-Expression on CD4+ Cells in Cyno PBMC

The TetraFab was next tested for its capacity to up-regulate ICAM-1 in a TNFR2-expressing primary T cell population (CD3+CD4+TIGIT+), as described below. ICAM-1 was selected because it is regulated by NF-KB, known to be a marker of acute T cell activation, and has a known role in Treg-mediated suppression. TIGIT was used to identify a population of mature T cells enriched for TNFR2 expression and Treg without having to stain for the intracellular marker FoxP3.

Briefly, 250 000 Human PBMC from various donors were plated in the wells of a 96-well plate in Optimizer CST medium. Each well was treated with 50 ml of 5 ng/ml IL-2 and 50 ml of 4× anti-TNFR2 antibody or TetraFab for 72 hours at 37° C. Subsequently, the cells were prepared for flow cytometry analysis. After 2 washes in PBS, the cells were first resuspended in 100 ml of Live Near IR staining buffer for 30 minutes at 4° C. After 2 additional washes in flow buffer, the cells were incubated with CD3 BV421, ICAM APC CD4 PE and TIGIT BUV395 diluted 1/40 1 hour at 4° C., then fixed with 1% PFA 15 minutes at room temperature (all reagents are summarized in table 13). Following 2 additional washes in flow buffer, the cells were finally analyzed by flow cytometry. Light scatter was used to exclude doublets, and low Near IR staining as an indication of viability. Single, viable cells were gated on the CD3+/CD4+/TIGIT+ population and ICAM-1 MFI measured for ICAM-1. A 4-parameter curve fit in Graph Pad Prism was used to calculate the EC50 of TNFR2 agonist for ICAM-1 induction. A similar strategy was used to determine activation of T cells in cyno PBMC, although T cell activation was measured as the percent double positive CD4+ cells for CD25+/Ki67+ because of differences in activation marker expression and antibody cross reactivity between human and cyno.

TABLE 13
reagents used for both human and cyno PBMC assays

| | Vendor | Catalog # | Clone |
|---|---|---|---|
| Human flow cytometry | | | |
| antibodies | | | |
| CD3 BV421 | BD Pharmingen | 562426 | UCHT1 |
| ICAM APC | BD Pharmingen | 559771 | HA58 (RUO) |
| CD4 PE | BD Pharmingen | 555347 | RPA-T4 |
| TIGIT BUV395 | BD OptiBuild | 747845 | 741182 |
| Cyno flow cytometry | | | |
| antibodies | | | |
| CD3 BUV395 | BD Pharmingen | 564088 | SP-34 |
| CD25 APC | eBioscience | 17-0257-42 | CD25-4E3 |
| CD4 FITC | BD Pharmingen | 550628 | L200 |
| Ki-67 PE-eFluor710 | Invitrogen | 46-5698-82 | SOLA15 |

As seen in the table 14 and similarly to the NF-KB-GFP Jurkat assay results, the EC50 for up-regulation of human ICAM-1 on the surface of human CD3+CD4+TIGIT+ cells is similar between the parental 854 antibody and the humanised 1765 antibody (1.402 and 1.308 mg/ml respectively). The EC50 for the up-regulation of ICAM-1 is 10-fold improved after treatment by the TetraFab 2053, confirming that increased valency will improve potency, even when the TNFR2 cell surface receptor number is low (11 000 in Jurkat, <500 in human Treg).

TABLE 14
Potency of the parental 854, its humanized counterpart 1765 and
the corresponding TetraFab 2053 in the human and cyno PBMC assays

| | EC50 HuPBMC | EC50 CyPBMC |
|---|---|---|
| Clone | μg/ml | μg/ml |
| 854 | 1.402 | 27.22 |
| 1765 | 1.308 | 13.63 |
| 2053 | 0.116 | 5.950 |

### Example 16 TetraFab 2053 Elicits TNFR2 Agonism-Induced IKBa Degradation in Cultured Human and Cyno Tregs

The following assay was used as a surrogate for NF-KB activation resulting from TNFR2 agonism. It also allowed a direct comparison of anti-TNFR2 antibodies pharmacology between human and cyno Tregs using the same endpoint.

Briefly, human or cyno natural Tregs sorted from the PBMC of various donors and expanded via multiple cycles of anti-CD3/28 stimulation in the presence of IL-2 and rapamycin were stimulated overnight at 37° C. in media containing 10 ng/ml of recombinant human or cyno IL2 respectively. The next day, the cells were seeded in 96 deep well plates (>50 000 cells/well) and were treated with various concentrations of TetraFab 2053, PMA/ionomycin (PMA 40 ng/ml; lonomycin 2 mM, positive control for IKBa) or media (un-stimulated control) for 20 minutes at 37° C. Following incubation, the cells were washed, fixed, permeabilized and stained with an anti-IKBa-PE antibody 30 minutes at 4° C. Finally, the cells were analyzed on a flow cytometer and degradation level of IKBa was assessed by MFI.

TABLE 15
potency of the TeraFab 2053 in the human
and cyno IKBa degradation assay.

| Human Donor# | Human EC50 | | Cyno Donor # | Cyno EC50 | |
|---|---|---|---|---|---|
| Human D28 | 20 | pM | Cyno 16-873 | 53.8 | pM |
| Human D32 | 37.3 | pM | Cyno3 | 2.248 | nM |
| Human D23 | 47.79 | pM | Cyno 16-477 | 117.7 | pM |
| Human D24 | 5.75 | pM | Cyno 18-7469 | 400 | pM |
| Human D28 | 17.9 | pM | Cyno 18-7473 | 155 | pM |

As detailed in table 15 above, the mean EC50 for IKBa in human expanded Tregs was 26+/−17 pM (n=5) while that for cyno expanded Tregs was 145+/−154 pM (n=5). Despite significant variability in results between Treg expanded from individual human and cyno donors of Treg, these data show consistent activation of the NF-KB pathway in Treg and a loss of potency in cyno of greater than 5-fold versus human.

### Example 17 Anti-TNFR2 TetraFab Induces OX-40 Up-Regulation in Human and Cyno Splenocytes in Absence of IL-2

Since pharmacology studies in cyno revealed activation of tissue Treg following in vivo administration as indicated by up-regulation of the TNF superfamily member OX-40, an ex vivo assay was set up to generate EC50 values for tissue resident Treg activation using TeraFab 2053. In this assay, frozen human or cyno splenocytes from various donors were thawed and treated with various concentrations of TetraFab 2053 for 22 hours at 37° C. Next, the samples were fixed, permeabilized and stained with the following antibodies: CD4-BV786, FoxP3-AF647, OX40-BV421, CD3-BV605, Tigit-A488, CD25-PE-Cy5, and CD45-PE-Cy7, before being analyzed by flow cytometry. Tissue resident Treg were identified as single/Live/CD45+/CD3+/CD4+/CD25+/FoxP3+ and the MFI of OX-40 used to generate EC50 curves in GraphPad Prism using a 4-parameter curve fit. Individual EC50 values generated for each donor of human or cyno frozen splenocytes are presented in this table. Table 16 summarizes the results obtained.

TABLE 16
Potency of TetraFab 2053 in inducing OX-40 up-regulation
in human and cyno splenocytes in absence of IL-2.

| | Human | | Cyno | |
|---|---|---|---|---|
| Human Donor# | EC50 | Cyno Donor# | EC50 | |
| ZenBio #072518 | 47 pM | TA1 | 642 | pM |
| BioIVT #BRH1515663 | 18 pM | 17-5215 | 1752 | pM |
| | | TA2 | 692 | pM |
| | | #31 | 799 | pM |
| | | #34 | 905 | pM |

The mean EC50 for OX-40 upregulation on human splenic Treg was 33+/−21 pM (n=2), similar to the EC50 for IKBa degradation in expanded human blood Treg, while that for upregulation of OX-40 in cyno splenic Treg was 958+/−455 pM (n=5).

### Example 18 In Vivo Pharmacology of TetraFab 2053 in Cynomolgus Monkeys

Male and female cynomolgus monkeys of Mauritius origin, aged greater than 2.5 years were acclimated for a minimum of 30 days prior to initiating dosing. Dose groups containing one male (M) and one female (F) animal were administered either vehicle control, or 20 mg/kg, 60 mg/kg, or 180 mg/kg of TetraFab 2053 by intravenous (IV) injection on Days 1, 4, 8, 11 and 15. Following administration of TetraFab 2053 on Day 11, blood was collected at various time points over 96 hours for toxicokinetic analysis. On Day 16, all animals were euthanized, and a portion of each spleen was collected for Treg phenotypic analysis. Single cell splenocyte suspensions were prepared by routine methods and leukocytes were then stained with panels of fluorescently labeled monoclonal antibodies recognizing surface and intracellular markers used to identify and characterize Treg. Stained cells were analyzed by flow cytometry for the following phenotypes:
OX40 expressing Treg: CD45+CD3+CD4+FoxP3+OX40+
TIGIT expressing Treg: CD45+CD3+CD4+FoxP3+TIGIT+
Proliferating Treg: CD45+CD3+CD4+FoxP3+Ki-67+

The percentages and mean fluorescence intensities (MFI) of OX40 or TIGIT expressing Treg, and the percentages of proliferating Treg were determined using flow cytometric analysis software. The fold-increases in Treg parameters in TetraFab 2053-treated animals compared to sex-matched vehicle controls are shown in the table 17 below. Total TetraFab 2053 concentrations in serum were determined using a ligand-binding assay and the Day 11-15 area under the curve (AUC) concentrations were assessed for each individual animal as shown below in table 17.

TABLE 17
summary of in vivo measurements made in cynomolgus monkeys
injected with various doses of TetraFab 2053

| | | | Fold-Increase Compared | | |
|---|---|---|---|---|---|
| | | | to Vehicle Control | | |
| In Vivo Measurement | | | 20 | 60 | 180 |
| Population | Parameter | Sex | mg/kg | mg/kg | mg/kg |
| OX40 | Percentage of | M | 1.66 | 1.83 | 1.68 |
| Expressing | FoxP3+ Treg | F | 1.93 | 3.00 | 3.14 |
| Treg | OX40 MFI | M | 1.43 | 1.32 | 1.08 |
| | | F | 2.23 | 2.46 | 1.90 |
| TIGIT | Percentage of | M | 1.64 | 1.27 | 1.76 |
| Expressing | FoxP3+ Treg | F | 1.67 | 1.63 | 1.75 |
| Treg | TIGIT MFI | M | 1.26 | 1.45 | 1.36 |
| | | F | 2.09 | 2.44 | 1.88 |
| | | M | 2.25 | 1.53 | 2.27 |
| Proliferating | Percentage of | F | 1.94 | 1.86 | 2.07 |
| Treg | FoxP3+ Treg | | | | |

| Serum Exposure | | AUC_{0-96 h} (μg*h/mL) | | |
|---|---|---|---|---|
| (Days 11-15) | Sex | 20 mg/kg | 60 mg/kg | 180 mg/kg |
| Total TetraFab 2053 | M | 5260 | 14100 | 64000 |
| | F | 11800 | 52500 | 175000 |

Administration of TetraFab 2053 to male and female cynomolgus monkeys at doses of to 180 mg/kg/dose IV for 16 days increased OX-40 and TIGIT expression on splenic Treg up to 3.14-fold and 2.44-fold, respectively, compared to vehicle treated controls, and increased the proportion of splenic Treg undergoing proliferation by as much as 2.27-fold. Increases in these markers of Treg activation were observed at all dose levels in both male and female animals and in general did not follow a dose-response relationship, most likely reflecting maximum pharmacologic activity.

Systemic exposure increased with increasing dose in an approximately dose-proportional manner from 20 to 60 mg/kg and greater than dose-proportional from 60 to 180 mg/kg in male cynomolgus monkeys. Systemic exposure in general increased with increasing dose in a greater than dose-proportional manner from 20 to 180 mg/kg in female cynomolgus monkeys.

### Example 19 TetraFab 2053 Shows Acceptable Developability

To confirm that TetraFab 2053 was a suitable candidate for further development as a biotherapeutic, a range of bioanalytical assays were completed (see table 18).

TABLE 18
summary of biophysical assays performed on
TeraFab 2053 to assess its developability

| Developability Assessment | | GBT-TNFR2-2053 |
|---|---|---|
| Categories | | (Tetrafab) |
| Non- | AC-SINS | acceptable |
| Specificity | DNA ELISA Score | acceptable |
| clearance/PK | Insulin ELISA Score | acceptable |
| | HuFcRn column rET | acceptable |
| Physical | Thermal stability | Acceptable |
| chemical | Low pH Hold | Acceptable @ pH 3.4 |
| | | (glycine) |
| properties | Viscosity | High but may be manageable |
| | | with buffer formulation |
| | High concentration | Acceptable stability at 4° C. |
| | stability | <5% increase % HMMS at |
| | 6 weeks at 4 and 25° C. | 25° C. |
| | Forced degradation | Acceptable |
| | study | |
| | 4 weeks at 40° C. | |

The results, summarized in the table above, showed that TetraFab 2053 displays acceptable non-specificity properties. Additionally, the molecule was stable when tested at high concentration for 6 weeks at 4° C. and 4 weeks at 40° C. Finally, TetraFab 2053 exhibited some viscosity at high concentration, but this undesirable feature may be managed with buffer formulation. All in all, these results suggested that TetraFab 2053 is suitable for manufacturing as a biotherapeutic.

### Example 16 Optimization of TetraFab 2053

The following amino acids shown in table 19 were targeted for optimization, to remove sequence liabilities (DE deamidation site, W oxidation site, predicted T-cell epitope) and decrease hydrophobicity.

TABLE 19
amino acid mutations introduced in TeraFab 2053 to remove
a range of sequence liability and decrease hydrophobicity

| Mutations (Kabat numbering) | purpose |
|---|---|
| Heavy Chain - W52K, V54Q | Hydrophobic patch and oxidation |
| Heavy Chain - W52R, V54E | site |
| Heavy Chain - W52F, G53Q, V54E | |
| Heavy Chain - W52L, V54H | |
| Heavy Chain - W52H, V54S | |
| Heavy Chain - W97S, D98T | Oxidation site and deamidation |
| | site |
| Heavy Chain - W97H | Oxidation site |
| Heavy Chain - W97R | |
| Heavy Chain - W97Q | |
| Light chain- S92N, Y94del, | Predicted T-cell epitope |
| P95del, L96Y, T97P | |
| Light chain- S92Y, Y94del, | |
| P95del, L96T, T97P | |
| Light chain- Q89L, Y91K | |
| Light chain- Q89L | |

Optimization constructs were made in IgG and TetraFab formats, and the various mutations described above were tested alone or in combination, for binding to Human TNFR2 CHO, as summarized in table 20:

TABLE 20
Binding to human TNFR2 CHO cells of the optimization
clones in IgG and TetraFab format.

| | | | | | TetraFab |
|---|---|---|---|---|---|
| | | | | IgG CHO | CHO |
| TetraFabs | IgGs | Heavy chain | Light Chain | binding | binding |
| 2083 | 1917 | W52K, V54Q | S92N, Y94del, P95del, | no | no |
| | | | L96Y, T97P | | |
| 2112 | 1926 | W52K, V54Q | S92Y, Y94del, P95del, | no | no |
| | | | L96T, T97P | | |
| 2141 | 1935 | W52K, V54Q | Q89L, Y91K | no | no |
| 2170 | 1944 | W52K, V54Q | Q89L | no | no |
| 2054 | 1908 | W52K, V54Q | parental | no | no |
| 2092 | 1973 | W52K, V54Q, W97S, D98T | S92N, Y94del, P95del, | no | no |
| | | | L96Y, T97P | | |
| 2121 | 1993 | W52K, V54Q, W97S, D98T | S92Y, Y94del, P95del, | no | no |
| | | | L96T, T97P | | |
| 2150 | 2013 | W52K, V54Q, W97S, D98T | Q89L, Y91K | no | no |
| 2179 | 2033 | W52K, V54Q, W97S, D98T | Q89L | no | no |
| 2063 | 1953 | W52K, V54Q, W97S, D98T | Parental | no | no |
| 2093 | 1974 | W52K, V54Q, W97H | S92N, Y94del, P95del, | no | no |
| | | | L96Y, T97P | | |
| 2122 | 1994 | W52K, V54Q, W97H | S92Y, Y94del, P95del, | no | no |
| | | | L96T, T97P | | |
| 2151 | 2014 | W52K, V54Q, W97H | Q89L, Y91K | no | no |
| 2180 | 2034 | W52K, V54Q, W97H | Q89L | no | no |
| 2064 | 1954 | W52K, V54Q, W97H | Parental | no | no |
| 2094 | 1975 | W52K, V54Q, W97R | S92N, Y94del, P95del, | no | no |
| | | | L96Y, T97P | | |
| 2123 | 1995 | W52K, V54Q, W97R | S92Y, Y94del, P95del, | no | no |
| | | | L96T, T97P | | |
| 2152 | 2015 | W52K, V54Q, W97R | Q89L, Y91K | no | no |
| 2181 | 2035 | W52K, V54Q, W97R | Q89L | no | no |
| 2065 | 1955 | W52K, V54Q, W97R | Parental | no | no |
| 2095 | 1976 | W52K, V54Q, W97Q | S92N, Y94del, P95del, | no | no |
| | | | L96Y, T97P | | |
| 2124 | 1996 | W52K, V54Q, W97Q | S92Y, Y94del, P95del, | no | no |
| | | | L96T, T97P | | |
| 2153 | 2016 | W52K, V54Q, W97Q | Q89L, Y91K | no | no |
| 2182 | 2036 | W52K, V54Q, W97Q | Q89L | no | no |
| 2066 | 1956 | W52K, V54Q, W97Q | Parental | no | no |
| 2084 | 1918 | W52R, V54E | S92N, Y94del, P95del, | no | no |
| | | | L96Y, T97P | | |
| 2113 | 1927 | W52R, V54E | S92Y, Y94del, P95del, | no | no |
| | | | L96T, T97P | | |
| 2142 | 1936 | W52R, V54E | Q89L, Y91K | no | no |
| 2171 | 1945 | W52R, V54E | Q89L | no | no |
| 2055 | 1909 | W52R, V54E | parental | no | no |
| 2096 | 1977 | W52R, V54E, W97S, D98T | S92N, Y94del, P95del, | no | no |
| | | | L96Y, T97P | | |
| 2125 | 1997 | W52R, V54E, W97S, D98T | S92Y, Y94del, P95del, | no | no |
| | | | L96T, T97P | | |
| 2154 | 2017 | W52R, V54E, W97S, D98T | Q89L, Y91K | no | no |
| 2183 | 2037 | W52R, V54E, W97S, D98T | Q89L | no | no |
| 2067 | 1957 | W52R, V54E, W97S, D98T | Parental | no | no |
| 2097 | 1978 | W52R, V54E, W97H | S92N, Y94del, P95del, | no | no |
| | | | L96Y, T97P | | |
| 2126 | 1998 | W52R, V54E, W97H | S92Y, Y94del, P95del, | no | no |
| | | | L96T, T97P | | |
| 2155 | 2018 | W52R, V54E, W97H | Q89L, Y91K | no | no |
| 2184 | 2038 | W52R, V54E, W97H | Q89L | no | no |
| 2068 | 1958 | W52R, V54E, W97H | Parental | no | no |
| 2098 | 1979 | W52R, V54E, W97R | S92N, Y94del, P95del, | no | no |
| | | | L96Y, T97P | | |
| 2127 | 1999 | W52R, V54E, W97R | S92Y, Y94del, P95del, | no | no |
| | | | L96T, T97P | | |
| 2156 | 2019 | W52R, V54E, W97R | Q89L, Y91K | no | no |
| 2185 | 2039 | W52R, V54E, W97R | Q89L | no | no |
| 2069 | 1959 | W52R, V54E, W97R | Parental | no | no |
| 2099 | 1980 | W52R, V54E, W97Q | S92N, Y94del, P95del, | no | no |
| | | | L96Y, T97P | | |
| 2128 | 2000 | W52R, V54E, W97Q | S92Y, Y94del, P95del, | no | no |
| | | | L96T, T97P | | |
| 2157 | 2020 | W52R, V54E, W97Q | Q89L, Y91K | no | no |
| 2186 | 2040 | W52R, V54E, W97Q | Q89L | no | no |
| 2070 | 1960 | W52R, V54E, W97Q | Parental | no | no |
| 2085 | 1919 | W52F, G53Q, V54E | S92N, Y94del, P95del, | no | no |
| | | | L96Y, T97P | | |
| 2114 | 1928 | W52F, G53Q, V54E | S92Y, Y94del, P95del, | no | no |
| | | | L96T, T97P | | |
| 2143 | 1937 | W52F, G53Q, V54E | Q89L, Y91K | no | no |
| 2172 | 1946 | W52F, G53Q, V54E | Q89L | no | no |
| 2056 | 1910 | W52F, G53Q, V54E | parental | no | no |
| 2100 | 1981 | W52F, G53Q, V54E, W97S, | S92N, Y94del, P95del, | no | no |
| | | D98T | L96Y, T97P | | |
| 2129 | 2001 | W52F, G53Q, V54E, W97S, | S92Y, Y94del, P95del, | no | no |
| | | D98T | L96T, T97P | | |
| 2158 | 2021 | W52F, G53Q, V54E, W97S, | Q89L, Y91K | no | no |
| | | D98T | | | |
| 2187 | 2041 | W52F, G53Q, V54E, W97S, | Q89L | no | no |
| | | D98T | | | |
| 2071 | 1961 | W52F, G53Q, V54E, W97S, | Parental | no | no |
| | | D98T | | | |
| 2101 | 1982 | W52F, G53Q, V54E, W97H | S92N, Y94del, P95del, | no | no |
| | | | L96Y, T97P | | |
| 2130 | 2002 | W52F, G53Q, V54E, W97H | S92Y, Y94del, P95del, | no | no |
| | | | L96T, T97P | | |
| 2159 | 2022 | W52F, G53Q, V54E, W97H | Q89L, Y91K | no | no |
| 2188 | 2042 | W52F, G53Q, V54E, W97H | Q89L | no | no |
| 2072 | 1962 | W52F, G53Q, V54E, W97H | Parental | no | no |
| 2102 | 1983 | W52F, G53Q, V54E, W97R | S92N, Y94del, P95del, | no | no |
| | | | L96Y, T97P | | |
| 2131 | 2003 | W52F, G53Q, V54E, W97R | S92Y, Y94del, P95del, | no | no |
| | | | L96T, T97P | | |
| 2160 | 2023 | W52F, G53Q, V54E, W97R | Q89L, Y91K | no | no |
| 2189 | 2043 | W52F, G53Q, V54E, W97R | Q89L | no | no |
| 2073 | 1963 | W52F, G53Q, V54E, W97R | Parental | no | no |
| 2103 | 1984 | W52F, G53Q, V54E, W97Q | S92N, Y94del, P95del, | no | no |
| | | | L96Y, T97P | | |
| 2132 | 2004 | W52F, G53Q, V54E, W97Q | S92Y, Y94del, P95del, | no | no |
| | | | L96T, T97P | | |
| 2161 | 2024 | W52F, G53Q, V54E, W97Q | Q89L, Y91K | no | no |
| 2190 | 2044 | W52F, G53Q, V54E, W97Q | Q89L | no | no |
| 2074 | 1964 | W52F, G53Q, V54E, W97Q | Parental | no | no |
| 2086 | 1920 | W52L, V54H | S92N, Y94del, P95del, | no | no |
| | | | L96Y, T97P | | |
| 2115 | 1929 | W52L, V54H | S92Y, Y94del, P95del, | no | no |
| | | | L96T, T97P | | |
| 2144 | 1938 | W52L, V54H | Q89L, Y91K | no | no |
| 2173 | 1947 | W52L, V54H | Q89L | no | no |
| 2057 | 1911 | W52L, V54H | parental | no | no |
| 2104 | 1985 | W52L, V54H, W97S, D98T | S92N, Y94del, P95del, | no | no |
| | | | L96Y, T97P | | |
| 2133 | 2005 | W52L, V54H, W97S, D98T | S92Y, Y94del, P95del, | no | no |
| | | | L96T, T97P | | |
| 2162 | 2025 | W52L, V54H, W97S, D98T | Q89L, Y91K | no | no |
| 2191 | 2045 | W52L, V54H, W97S, D98T | Q89L | no | no |
| 2075 | 1965 | W52L, V54H, W97S, D98T | Parental | no | no |
| 2105 | 1986 | W52L, V54H, W97H | S92N, Y94del, P95del, | no | no |
| | | | L96Y, T97P | | |
| 2134 | 2006 | W52L, V54H, W97H | S92Y, Y94del, P95del, | no | no |
| | | | L96T, T97P | | |
| 2163 | 2026 | W52L, V54H, W97H | Q89L, Y91K | no | no |
| 2192 | 2046 | W52L, V54H, W97H | Q89L | no | no |
| 2076 | 1966 | W52L, V54H, W97H | Parental | no | no |
| 2106 | 1987 | W52L, V54H, W97R | S92N, Y94del, P95del, | no | no |
| | | | L96Y, T97P | | |
| 2135 | 2007 | W52L, V54H, W97R | S92Y, Y94del, P95del, | no | no |
| | | | L96T, T97P | | |
| 2164 | 2027 | W52L, V54H, W97R | Q89L, Y91K | no | no |
| 2193 | 2047 | W52L, V54H, W97R | Q89L | no | no |
| 2077 | 1967 | W52L, V54H, W97R | Parental | no | no |
| 2107 | 1988 | W52L, V54H, W97Q | S92N, Y94del, P95del, | no | no |
| | | | L96Y, T97P | | |
| 2136 | 2008 | W52L, V54H, W97Q | S92Y, Y94del, P95del, | no | no |
| | | | L96T, T97P | | |
| 2165 | 2028 | W52L, V54H, W97Q | Q89L, Y91K | no | no |
| 2194 | 2048 | W52L, V54H, W97Q | Q89L | no | no |
| 2078 | 1968 | W52L, V54H, W97Q | Parental | no | no |
| 2087 | 1921 | W52H, V54S | S92N, Y94del, P95del, | no | no |
| | | | L96Y, T97P | | |
| 2116 | 1930 | W52H, V54S | S92Y, Y94del, P95del, | no | no |
| | | | L96T, T97P | | |
| 2145 | 1939 | W52H, V54S | Q89L, Y91K | no | no |
| 2174 | 1948 | W52H, V54S | Q89L | no | no |
| 2058 | 1912 | W52H, V54S | parental | no | no |
| 2108 | 1989 | W52H, V54S, W97S, D98T | S92N, Y94del, P95del, | no | no |
| | | | L96Y, T97P | | |
| 2137 | 2009 | W52H, V54S, W97S, D98T | S92Y, Y94del, P95del, | no | no |
| | | | L96T, T97P | | |
| 2166 | 2029 | W52H, V54S, W97S, D98T | Q89L, Y91K | no | no |
| 2195 | 2049 | W52H, V54S, W97S, D98T | Q89L | no | no |
| 2079 | 1969 | W52H, V54S, W97S, D98T | Parental | no | no |
| 2109 | 1990 | W52H, V54S, W97H | S92N, Y94del, P95del, | no | no |
| | | | L96Y, T97P | | |
| 2138 | 2010 | W52H, V54S, W97H | S92Y, Y94del, P95del, | no | no |
| | | | L96T, T97P | | |
| 2167 | 2030 | W52H, V54S, W97H | Q89L, Y91K | no | no |
| 2196 | 2050 | W52H, V54S, W97H | Q89L | no | no |
| 2080 | 1970 | W52H, V54S, W97H | Parental | no | no |
| 2110 | 1991 | W52H, V54S, W97R | S92N, Y94del, P95del, | no | no |
| | | | L96Y, T97P | | |
| 2139 | 2011 | W52H, V54S, W97R | S92Y, Y94del, P95del, | no | no |
| | | | L96T, T97P | | |
| 2168 | 2031 | W52H, V54S, W97R | Q89L, Y91K | no | no |
| 2197 | 2051 | W52H, V54S, W97R | Q89L | no | no |
| 2081 | 1971 | W52H, V54S, W97R | Parental | no | no |
| 2111 | 1992 | W52H, V54S, W97Q | S92N, Y94del, P95del, | no | no |
| | | | L96Y, T97P | | |
| 2140 | 2012 | W52H, V54S, W97Q | S92Y, Y94del, P95del, | no | no |
| | | | L96T, T97P | | |
| 2169 | 2032 | W52H, V54S, W97Q | Q89L, Y91K | no | no |
| 2198 | 2052 | W52H, V54S, W97Q | Q89L | no | no |
| 2082 | 1972 | W52H, V54S, W97Q | Parental | no | no |
| 2088 | 1922 | W97S, D98T | S92N, Y94del, P95del, | no | no |
| | | | L96Y, T97P | | |
| 2117 | 1931 | W97S, D98T | S92Y, Y94del, P95del, | no | no |
| | | | L96T, T97P | | |
| 2146 | 1940 | W97S, D98T | Q89L, Y91K | no | no |
| 2175 | 1949 | W97S, D98T | Q89L | no | no |
| 2059 | 1913 | W97S, D98T | parental | no | no |
| 2089 | 1923 | W97H | S92N, Y94del, P95del, | no | no |
| | | | L96Y, T97P | | |
| 2118 | 1932 | W97H | S92Y, Y94del, P95del, | no | no |
| | | | L96T, T97P | | |
| 2147 | 1941 | W97H | Q89L, Y91K | no | no |
| 2176 | 1950 | W97H | Q89L | no | no |
| 2060 | 1914 | W97H | parental | no | no |
| 2090 | 1924 | W97R | S92N, Y94del, P95del, | no | no |
| | | | L96Y, T97P | | |
| 2119 | 1933 | W97R | S92Y, Y94del, P95del, | no | no |
| | | | L96T, T97P | | |
| 2148 | 1942 | W97R | Q89L, Y91K | no | no |
| 2177 | 1951 | W97R | Q89L | no | no |
| 2061 | 1915 | W97R | parental | no | no |
| 2091 | 1925 | W97Q | S92N, Y94del, P95del, | no | no |
| | | | L96Y, T97P | | |
| 2120 | 1934 | W97Q | S92Y, Y94del, P95del, | no | no |
| | | | L96T, T97P | | |
| 2149 | 1943 | W97Q | Q89L, Y91K | no | no |
| 2178 | 1952 | W97Q | Q89L | no | no |
| 2062 | 1916 | W97Q | parental | no | no |
| 2206 | 2202 | Parental | S92N, Y94del, P95del, | no | no |
| | | | L96Y, T97P | | |
| 2207 | 2203 | Parental | S92Y, Y94del, P95del, | no | no |
| | | | L96T, T97P | | |
| 2208 | 2204 | Parental | Q89L, Y91K | 1.63 nM | 2.30 nM |
| 2209 | 2205 | Parental | Q89L | 0.43 nM | 0.15 nM |
| 2053 | 1765 | Parental | parental | 0.67 nM | 0.70 nM |

Surprisingly, all clones, except 2 pairs of IgGs and TetraFabs, completely lost their ability to bind to human TNFR2 expressed on the surface of CHO cells. Clones TetraFab 2208/IgG 2204 and TetraFab 2209/IgG2205 were still able to bind to TNFR2 as IgG and TetraFab, but the Emax was significantly decreased compared to the parental clones 2053/1765. These clones were only mutated in the CDR3-VL, removing a predicted T cell epitope. In vitro analysis (not shown) showed that TetraFab 2053 predicted T cell epitope is not presented on the surface of Antigen Presenting Cells, and a peptide overlapping the predicted T cell epitope doesn't activate CD4+ T-cells, suggesting the risk of immunogenicity brought on by that predicted T cell epitope is low, and therefore these optimization variants were not brought forward.

### Example 17 TF-2053 Provides a Long-Term Survival Benefit in the NSG-GVHD Model

### Objective: To Test Prophylactic Efficacy of Human TetraFab-2053 in Preventing Xenograft GvHD

Transfer of human peripheral blood mononuclear cells (PBMC) into immunodeficient mice devoid of endogenous lymphoid cell differentiation (NOD-SCID gamma common chain knockout, NSG) results in graft versus host disease (GVHD) mediated predominately by the transferred human CD4+ and CD8+ lymphocytes[1]. The transferred human lymphocytes do not undergo thymic education in the recipient mice, and thus are not tolerized to mouse proteins presented in the context of mouse major histocompatibility locus (MHC) class I and II molecules[2]. Mouse-reactive human CD4+ and CD8+ cells mature and proliferate over the course of weeks into effector memory cells that release copious amounts of inflammatory cytokines like tumor necrosis factor alpha (TNFa) and interferon gamma (IFNg), which, in addition to extensive tissue inflammatory infiltrate, results in organ disfunction, body weight loss and death[1]. The NSG GVHD model is often used by the field to demonstrate the role of proteins expressed by human lymphocytes in mediating T cell activation, expansion, and effector functions. This model was chosen in order to demonstrate the role of agonizing TNFR2 in reducing the expansion, activation, and pathophysiologic function of human effector T cells in a model independent of human regulatory T cell (Treg) survival. Effector T cell activation-induced cell death via TNFR2 agonism has previously been demonstrated in vitro [3]but not for in vivo for human T cells, although it has been documented in the mouse that TNFR2 is responsible for restraining some autoreactive CD8 T cell activities[4].

### Methods:

Summary: 8-9 week-old NSG mice were purchased from Jackson Labs and acclimatized in the Pfizer vivarium. All animal use and handling were done under IACUC-approved protocols. TetraFab-2053 or an isotype control tetraFab were administered at 3 mg/kg, 10 ml/kg biweekly throughout the experiment, starting 1 day prior to human PBMC administration. Previously cryopreserved human PBMC were thawed on day 0 of the experiment, washed in PBS and administered IV at 10×10″6/0.1 ml PBS per mouse, n=20 mice per group. Cohorts were formed based on randomization of pre-study body weights. Body weights were measured 2× per week until sacrifice on Day 78. Blood was collected at 2-week intervals to measure human cell engraftment/expansion, CD4/CD8 distribution, as well as markers of T-cell anergy, exhaustion and senescence by flow cytometry and circulating human cytokine levels by electrochemiluminescent MSD assay. Mice that lost more than 20% BW were euthanized per IACUC protocol and all remaining mice terminated at Day 78. At termination, spleen and liver weights were recorded and flow cytometric analysis performed on splenocytes as well as blood.

### Results:

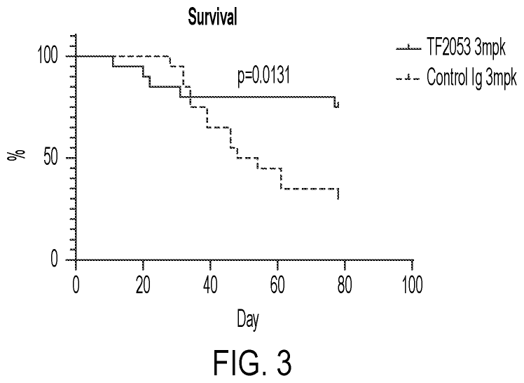

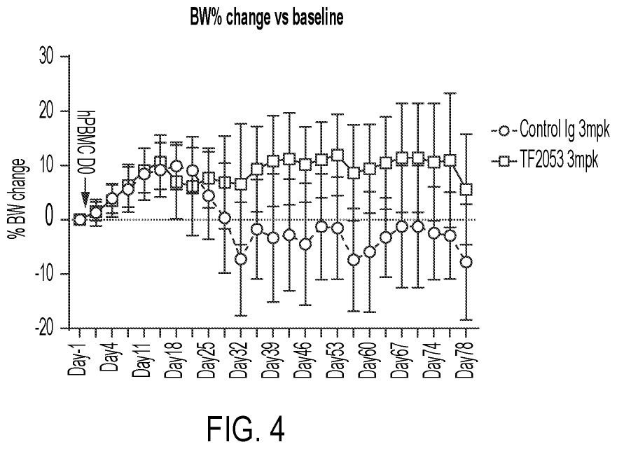

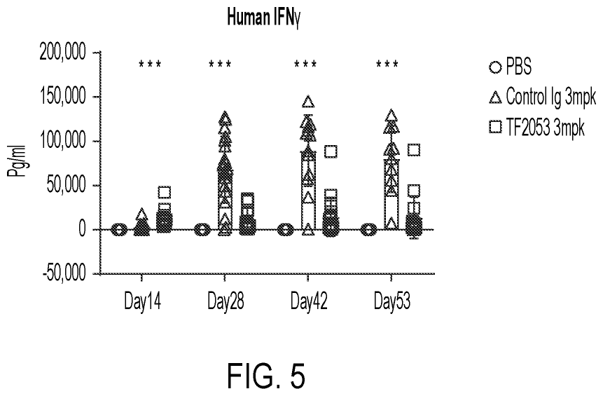

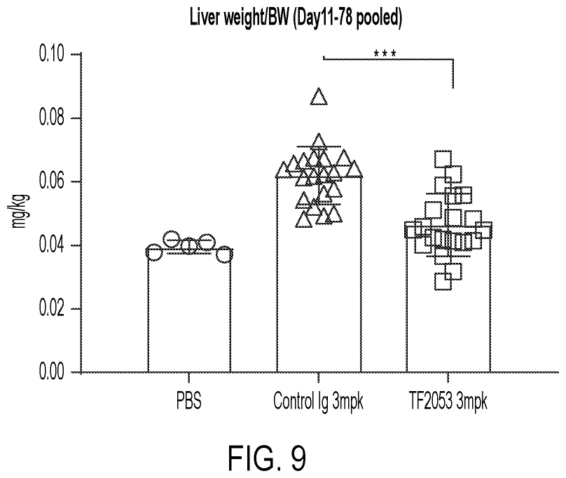

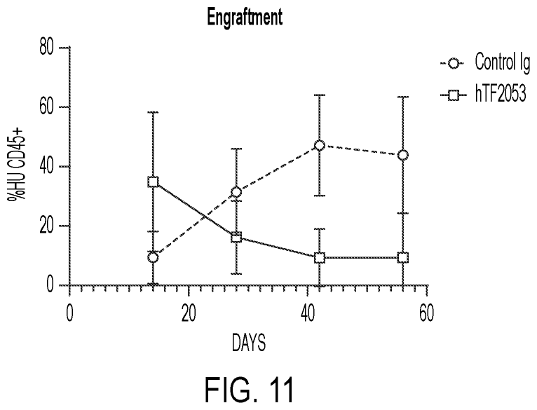

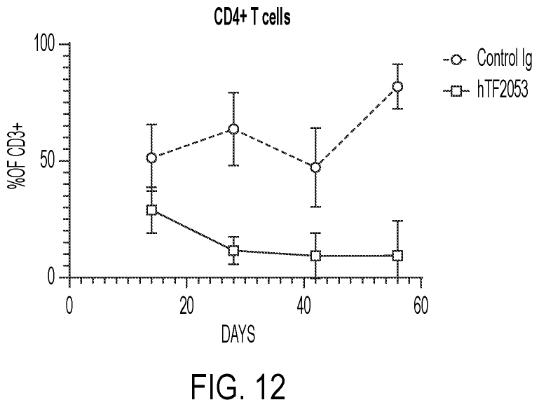

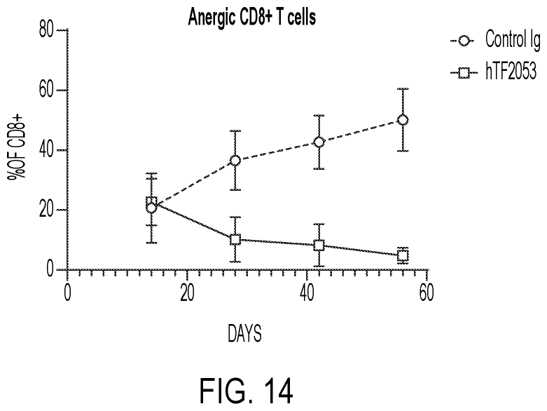

TF2053 provides a long-term survival benefit in the NSG-GVHD model (FIG. 3, Table 21). TF2053 reduces body-weight loss in NSG-GVHD model (FIG. 4, Table 22). Further, in the NSG GVHD model TF2053 reduces circulating human cytokines IFNγ, 11_10, I L17A, TNFα (FIGS and Tables 23-26). TF2053 reduces liver and spleen weight at sacrifice in the NSG GVHD model (FIGS. 9 & 10, Tables 27 & 28). TF2053 reduces engraftment of human cells over the course of the NSG GVHD model as measured by CD45+, CD4+, and CD8+, (FIGS. 11-13, Table s27-29). TF2053 reduces anergic and increases senescent and exhausted CD8+ cells over the course of the NSG GVHD model (FIGS. 14-16, Tables 32-34). TF2053 changes CD4:CD8 engraftment ratio (FIG. 12, Table 30).

TABLE 21
Survival at Day 78 (% of starting population).

| PBS (no PBMC) | Control Ig | TetraFab-2053 |
|---|---|---|
| 100 | 30 | 75 |

Statistics: Log-rank (Mantel-Cox) test. Data also shown in FIG. 3.

TABLE 22
Body Weight percentage change versus baseline.

| PBS (no PBMC) | Control Ig | TF2053 |
|---|---|---|
| 10.0 | −7.8 | 5.6 |

Data also shown in FIG. 4.

TABLE 23
Mean Plasma human IFNγ (pg/ml).

| | | Control Ig | TF2053 | |
|---|---|---|---|---|
| | PBS | 3 mpk | 3 mpk | |
| Day 14 | 0 | 2719 | 11734 | *** |
| Day 28 | 0 | 67924 | 12201 | *** |
| Day 42 | 0 | 89370 | 14523 | *** |
| Day 53 | 0 | 80091 | 13457 | *** |

*** p < 0.001 TF2053 vs Control Ig. Data also shown in FIG. 5.

TABLE 24
Mean plasma human IL10 (pg/ml).

| | | Control Ig | TF2053 | |
|---|---|---|---|---|
| | PBS | 3 mpk | 3 mpk | |
| Day 14 | 0.1 | 1.7 | 1.0 | |
| Day 28 | 0.1 | 13.8 | 0.6 | *** |
| Day 42 | 0.0 | 9.9 | 0.9 | *** |
| Day 53 | 0.0 | 8.2 | 0.8 | *** |

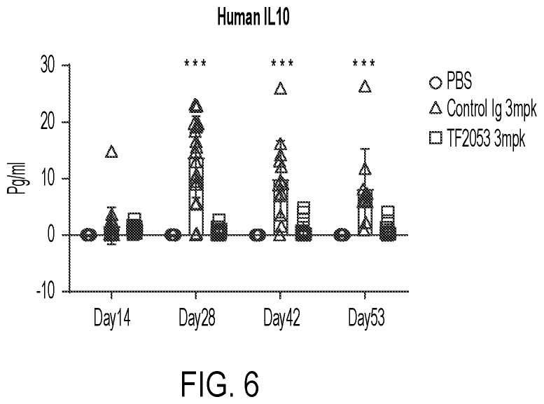

*** p < 0.001 TF2053 vs Control Ig. Data also shown in FIG. 6.

TABLE 25
Mean plasma human IL17A (pg/ml).

| | | Control Ig | TF2053 | |
|---|---|---|---|---|
| | PBS | 3 mpk | 3 mpk | |
| Day 14 | 0 | 3 | 6 | |
| Day 28 | 0 | 132 | 26 | ** |
| Day 42 | 0 | 636 | 30 | *** |
| Day 53 | 1 | 707 | 34 | *** |

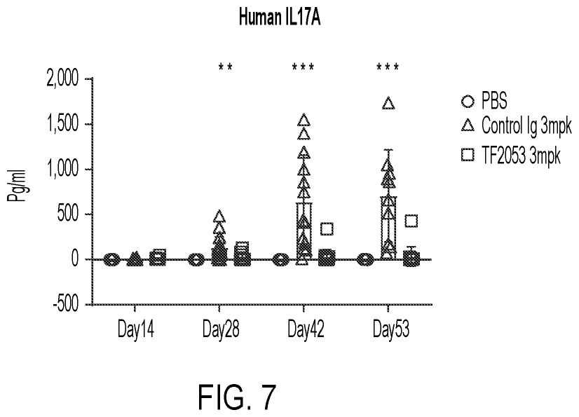

** p < 0.01 TF2053 vs Control Ig.
*** p < 0.001 F2053 vs Control Ig. Data also shown in FIG. 7.

TABLE 26
Mean plasma human TNFa (pg/ml).

| | | Control Ig | TF2053 | |
|---|---|---|---|---|
| | PBS | 3 mpk | 3 mpk | |
| Day 14 | 0.0 | 1.2 | 4.2 | *** |
| Day 28 | 0.0 | 26.7 | 5.0 | *** |
| Day 42 | 0.2 | 17.6 | 4.0 | *** |
| Day 53 | 0.0 | 16.0 | 3.9 | *** |

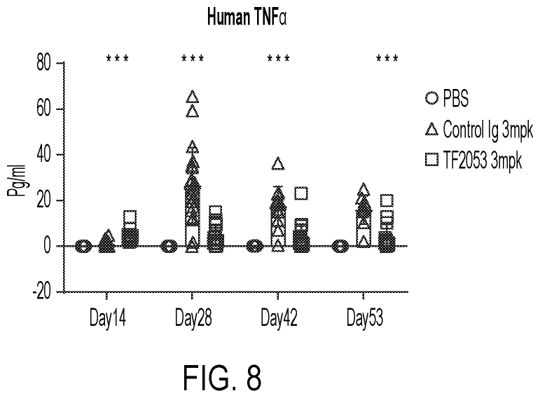

*** p < 0.001 TF2053 vs Control Ig. Statistics: unpaired 2-tailed Student's t-Test. Data also shown in FIG. 8.

TABLE 27
Mean liver weight per body weight at sacrifice (mg/kg).

| | | Control Ig | TF2053 | |
|---|---|---|---|---|
| | PBS | 3 mpk | 3 mpk | |
| Day 11-78 | 0.0395 | 0.0620 | 0.0465 | *** |

*** p < 0.001 TF2053 vs Control Ig. Data also shown in FIG. 9.

TABLE 28
Mean spleen weight per body weight at sacrifice (mg/kg).

| | | Control Ig | TF2053 | |
|---|---|---|---|---|
| | PBS | 3 mpk | 3 mpk | |
| Day 11-78 | 0.0009 | 0.0061 | 0.0027 | *** |

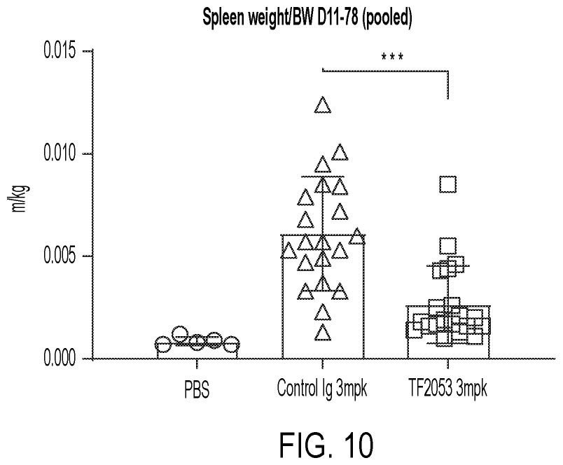

*** p < 0.001 TF2053 vs Control Ig. Statistics: unpaired 2-tailed Student's t-Test. Data also shown in FIG. 10.

TABLE 29
Mean percent human CD45+ cells in whole blood leukocytes.

| | Control Ig | TF2053 | |
|---|---|---|---|
| | 3 mpk | 3 mpk | |
| Day 14 | 1.2 | 4.2 | *** |
| Day 28 | 26.7 | 4.9 | ** |
| Day 42 | 17.6 | 4.0 | **** |
| Day 53 | 16.0 | 3.9 | *** |

** p < 0.01.
*** p < 0.001.
**** p < 0.0001. Data also shown in FIG. 11

TABLE 30
Mean percent human CD4+ of human CD45+/CD3+
whole blood lymphocytes.

| | Control Ig | TF2053 | |
|---|---|---|---|
| | 3 mpk | 3 mpk | |
| Day 14 | 51.3 | 28.9 | **** |
| Day 28 | 63.6 | 11.6 | **** |
| Day 42 | 47.2 | 9.3 | **** |
| Day 53 | 81.9 | 9.4 | **** |

**** p < 0.0001. Data also shown in FIG. 12.

TABLE 31
Mean percent human CD8+ of human CD45+/CD3+
whole blood lymphocytes.

| | Control Ig | TF2053 | |
|---|---|---|---|
| | 3 mpk | 3 mpk | |
| Day 14 | 44.4 | 66.0 | **** |
| Day 28 | 35.5 | 87.6 | **** |
| Day 42 | 23.9 | 74.6 | **** |
| Day 53 | 16.1 | 72.1 | **** |

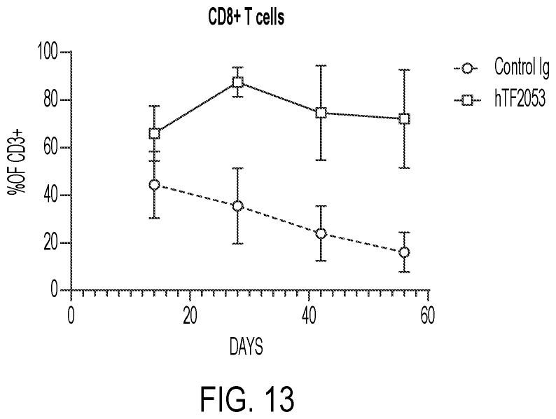

**** p < 0.0001. Data also shown in FIG. 13.

TABLE 32
Mean percent anergic (PD1+ KLRG1− CD57−) cells of human
CD8+/CD3+/CD45+ blood lymphocytes.

| | Control Ig | TF2053 | |
|---|---|---|---|
| | 3 mpk | 3 mpk | |
| Day 14 | 44.4 | 66.0 | |
| Day 28 | 35.5 | 87.6 | **** |
| Day 42 | 23.9 | 74.6 | **** |
| Day 53 | 16.1 | 72.1 | **** |

**** p < 0.0001. Data also shown in FIG. 14.

TABLE 33
Mean percent exhausted (PD1+ KLRG1+ CD57−) cells of
human CD8+/CD3+/CD45+ blood lymphocytes.

| | Control Ig | TF2053 | |
|---|---|---|---|
| | 3 mpk | 3 mpk | |
| Day 14 | 30.4 | 43.7 | ** |
| Day 28 | 41.7 | 43.5 | |
| Day 42 | 33.8 | 45.2 | ** |
| Day 53 | 31.9 | 44.5 | ** |

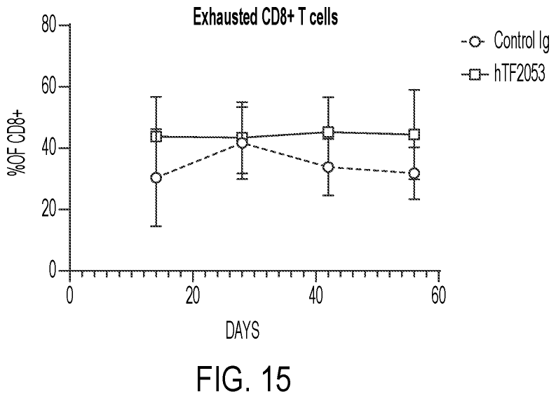

** p < 0.01. Data also shown in FIG. 15.

TABLE 34
Mean percent senescent (PD1− KLRG1+ CD57+) cells of
human CD8+/CD3+/CD45+ blood lymphocytes.

| | Control Ig | TF2053 | |
|---|---|---|---|
| | 3 mpk | 3 mpk | |
| Day 14 | 13.6 | 1.0 | * |
| Day 28 | 0.5 | 4.2 | **** |
| Day 42 | 1.0 | 5.7 | **** |
| Day 53 | 0.7 | 9.0 | ** |

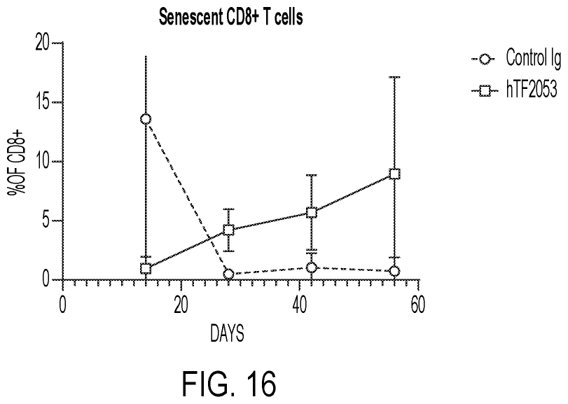

* p < 0.05.
** p < 0.01.
**** p < 0.0001. Statistics: unpaired 2-tailed T test between groups at each timepoint. Data also shown in FIG. 16.

### Conclusion:

Treatment of NSG mice with 3 mg/kg TF2053 beginning one day prior to transfer of 10″7 human PBMC provided significant protection from GVHD. Survival was improved, while body weight loss was reduced. In addition, increases in liver and spleen weight were reduced. Circulating human inflammatory cytokines and human CD45 cell engraftment were significantly higher at Day 14 but significantly lower in the TF2053 at all later timepoints. TF2053 also altered the CD4 to CD8 ratio, favoring CD8+ cells, and increased the percentage of these CD8+ cells that were exhausted and senescent over time while reducing the percent anergic. In summary, the hypothesis that agonism of TNFR2 could provide a benefit in the humanized NSG-GVHD in vivo model via effects on effector T cells in the absence of regulatory T cell expansion was confirmed.

### REFERENCES

1. Pino, S., et al., Development of novel major histocompatibility complex class I and class II-deficient NOD-SCID IL2R gamma chain knockout mice for modeling human xenogeneic graft-versus-host disease. Methods Mol Biol, 2010. 602: p. 105-17.
2. Brehm, M. A., et al., Lack of acute xenogeneic graft-versus-host disease, but retention of T-cell function following engraftment of human peripheral blood mononuclear cells in NSG mice deficient in MHC class I and II expression. Faseb j, 2019. 33 (3): p. 3137-3151
3. Ban, L., et al., Selective death of autoreactive T cells in human diabetes by TNF or TNF receptor 2 agonism. Proc Natl Acad Sci USA, 2008. 105 (36): p. 13644-9.
4. Punit, S., et al., Tumor Necrosis Factor Receptor 2 Restricts the Pathogenicity of CD8(+) T Cells in Mice With Colitis. Gastroenterology, 2015. 149 (4): p. 993-1005.e2.

TABLE 35
SEQUENCES LIST

| SEQ | Description | Sequence |
|---|---|---|
| 1 | GBT-0854 VL CDR-L1 | KASQNVGTAVA |
| | GBT-1765 VL CDR-L1 | |
| | GBT-2053 VL CDR-L1 | |
| 2 | GBT-0854 VL CDR-L2 | SASNRYT |
| 3 | GBT-0854 VL CDR-L3 | QQYSSYPLT |
| | GBT-1765 VL CDR-L3 | |
| | GBT-2053 VL CDR-L3 | |
| 4 | GBT-0854 VL | DIVMTQSQKF MSTTVGDRVS ITCKASQNVG TAVAWYQQKP GQSPKLLIYS |
| | | ASNRYTGVPD RFTGSGSGTD FTLTISNMQS EDLADYFCQQ YSSYPLTFGG |
| | | GTKLEIK |
| 5 | Constant domain | RTVAAPSVFI FPPSDEQLKS GTASVVCLLN NFYPREAKVQ WKVDNALQSG |
| | light chain of | NSQESVTEQD SKDSTYSLSS TLTLSKADYE KHKVYACEVT HQGLSSPVTK |
| | HuKappa | SFNRGEC |
| 6 | GBT-0854 LC | DIVMTQSQKF MSTTVGDRVS ITCKASQNVG TAVAWYQQKP GQSPKLLIYS |
| | | ASNRYTGVPD RFTGSGSGTD FTLTISNMQS EDLADYFCQQ YSSYPLTFGG |
| | | GTKLEIKRTV AAPSVFIFPP SDEQLKSGTA SVVCLLNNFY PREAKVQWKV |
| | | DNALQSGNSQ ESVTEQDSKD STYSLSSTLT LSKADYEKHK VYACEVTHQG |
| | | LSSPVTKSEN RGEC |
| 7 | GBT-1765 VL CDR-L2 | AASTLQS |
| | GBT-2053 VL CDR-L2 | |
| 8 | GBT-1765 VL | DIQLTQSPSF LSASVGDRVT ITCKASQNVG TAVAWYQQKP GKAPKLLIYA |
| | | ASTLQSGVPS RFSGSGSGTE FTLTISSLQP EDFATYYCQQ YSSYPLTFGG |
| | | GTKVEIK |
| 9 | GBT-1765 LC | DIQLTQSPSF LSASVGDRVT ITCKASQNVG TAVAWYQQKP GKAPKLLIYA |
| | GBT-2053 LC | ASTLQSGVPS RFSGSGSGTE FTLTISSLQP EDFATYYCQQ YSSYPLTFGG |
| | | GTKVEIKRTV AAPSVFIFPP SDEQLKSGVT ASVVCLLNNF YPREAKVQWK |
| | | VDNALQSGNS QESVTEQDSK DSTYSLSSTL TLSKADYEKH KVYACEVTHQ |
| | | GLSSPVTKSF NRGEC |
| 10 | GBT-0854 VH CDR-H1 | GFSFPSSGVD |
| | GBT-1765 VH CDR-H1 | |
| | GBT-2053 VH CDR-H1 | |
| 11 | GBT-0854 VH CDR-H2 | VTWGVGSTHY NSALKS |
| 12 | GBT-0854 VH CDR-H3 | GEWDEGFVY |
| | GBT-1765 VH CDR-H3 | |
| | GBT-2053 VH CDR-H3 | |
| 13 | GBT-0854 VH | QVQLKESGPG LVAPSQSLSI TCTVSGFSFP SSGVDWVRQS PGKGLEWLGV |
| | | TWGVGSTHYN SALKSRLSIS KDNSKSQVFL KMNSLQTEDT AIYYCASGEW |
| | | DEGFVYWGQG TLVTVSA |
| 14 | IgG2 CH1 | ASTKGPSVFP LAPCSRSTSE STAALGCLVK DYFPEPVTVS WNSGALTSGV |
| | | HTFPAVLQSS GLYSLSSVVT VPSSNEGTQT YTCNVDHKPS NTKVDKTV |
| 15 | IgG2 Hinge | ERKCCVECPPCP |
| 16 | IgG2 CH2 | APPVAGPSVF LFPPKPKDTL MISRTPEVTC VVVAVSHEDP EVQFNWYVDG |
| | | VEVHNAKTKP REEQFNSTFR VVSVLTVVHQ DWLNGKEYKC KVSNKGLPSS |
| | | IEKTISKTK |
| 17 | IgG2 CH3 | GQPREPQVYT LPPSREEMTK NQVSLTCLVK GFYPSDIAVE WESNGQPENN |
| | | YKTTPPMLDS DGSFFLYSKL TVDKSRWQQG NVFSCSVMHE ALHNHYTQKS |
| | | LSLSPGK |
| 18 | Fc chain of HuIgG2 | ERKCCVECPP CPAPPVAGPS VFLFPPKPKD TLMISRTPEV TCVVVAVSHE |
| | effector function | DPEVQFNWYV DGVEVHNAKT KPREEQFNST FRVVSVLTVV HQDWLNGKEY |
| | null | KCKVSNKGLP SSIEKTISKT KGQPREPQVY TLPPSREEMT KNQVSLTCLV |
| | Hinge in italics | KGFYPSDIAV EWESNGQPEN NYKTTPPMLD SDGSFFLYSK LTVDKSRWQQ |
| | | GNVFSCSVMH EALHNHYTQK SLSLSPGK |
| 19 | GBT-0854 HC | QVQLKESGPG LVAPSQSLSI TCTVSGFSFP SSGVDWVRQS PGKGLEWLGV |
| | | TWGVGSTHYN SALKSRLSIS KDNSKSQVFL KMNSLQTEDT AIYYCASGEW |
| | | DEGFVYWGQG TLVTVSAAST KGPSVFPLAP CSRSTSESTA ALGCLVKDYF |
| | | PEPVTVSWNS GALTSGVHTF PAVLQSSGLY SLSSVVTVPS SNFGTQTYTC |
| | | NVDHKPSNTK VDKTVERKCC VECPPCPAPP VAGPSVFLFP PKPKDTLMIS |
| | | RTPEVTCVVV AVSHEDPEVQ FNWYVDGVEV HNAKTKPREE QFNSTFRVVS |
| | | VLTVVHQDWL NGKEYKCKVS NKGLPSSIEK TISKTKGQPR EPQVYTLPPS |
| | | REEMTKNQVS LTCLVKGFYP SDIAVEWESN GQPENNYKTT PPMLDSDGSF |
| | | FLYSKLTVDK SRWQQGNVFS CSVMHEALHN HYTQKSLSLS PGK |
| 20 | GBT-1765 VH CDR-H2 | VTWGVGSTHY NQKFQG |
| | GBT-2053 VH CDR-H2 | |
| 21 | GBT-1765 VH | QVQLVQSGAE VKKPGASVKV SCKVSGFSFP SSGVDWVRQA PGQGLEWLGV |
| | | TWGVGSTHYN QKFQGRVTIT KDTSKSTVYM ELSSLRSEDT AVYYCASGEW |
| | | DEGFVYWGQG TLVTVSS |
| 22 | GBT-1765 HC | QVQLVQSGAEVKKPGASVKVSCKVSGFSFPSSGVDWVRQA PGQGLEWLGV |
| | | TWGVGSTHYNQKFQGRVTITKDTSKSTVYM ELSSLRSEDT AVYYCASGEW |
| | | DEGFVYWGQGTLVTVSSASTKGPSVFPLAPCSRSTSESTAALGCLVKDYFPEPVTVS |
| | | WNSGALTSGVHTFPAVLQSSGLYSLSSVVTVPSSNFGTQTYTCNVDHKPSNTKVDKT |
| | | VERKCCVECPPCPAPPVAGPSVFLFPPKPKDTLMISRTPEVTCVVVAVSHEDPEVQF |
| | | NWYVDGVEVHNAKTKPREEQFNSTFRVVSVLTVVHQDWLNGKEYKCKVSNKGLPSSI |
| | | EKTISKTKGQPREPQVYTLPPSREEMTKNQVSLTCLVKGFYPSDIAVEWESNGQPEN |
| | | NYKTTPPMLDSDGSFFLYSKLTVDKSRWQQGNVFSCSVMHEALHNHYTQKSLSLSPG |
| | | K |
| 23 | IgG1 CH1 | ASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSC |
| 24 | IgG1 Hinge | DKTHTCPPCP |
| 25 | IgG1 CH2 | APEAAGAPSVFLFPPKPKDTLMISRTPEVTCVVVDVSHEDPEVKFNWYVDGVEVHNA |
| | effector mutations | KTKPREEQYNSTYRVVSVLTVLHQDWLNGKEYKCKVSNKALPAPIEKTISKAK |
| | bold & underlined | |
| 26 | IgG1 CH3 | GQPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 27 | Linker | [symbol] |
| 28 | IgG1 CH1 and | ASTKGPSVFPLAPSSKSTSGGTAALGCLVKDYFPEPVTVSWNSGALTSGVHTFPAVL |
| | Linker | QSSGLYSLSSVVTVPSSSLGTQTYICNVNHKPSNTKVDKKVEPKSCGGGGS |
| 29 | GBT-2053 VH | QVQLVQSGAEVKKPGASVKVSCKVSGFSFPSSGVDWVRQAPGQGLEWLGVTWGVGST |
| | | HYNQKFQGRVTITKDTSKSTVYMELSSLRSEDTAVYYCASGEWDEGFVYWGQGTLVT |
| | | VSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPKSC [symbol] QVQ |
| | | LVQSGAEVKKPGASVKVSCKVSGFSFPSSGVDWVRQAPGQGLEWLGVTWGVGSTHYN |
| | | QKFQGRVTITKDTSKSTVYMELSSLRSEDTAVYYCASGEWDEGFVYWGQGTLVTVSS |
| 30 | GBT-2053 dual Fab | QVQLVQSGAEVKKPGASVKVSCKVSGFSFPSSGVDWVRQAPGQGLEWLGVTWGVGST |
| | HC | HYNQKFQGRVTITKDTSKSTVYMELSSLRSEDTAVYYCASGEWDEGFVYWGQGTLVT |
| | | VSSASTKGPSVFPLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVEPKSC |
| | | [symbol] QVQLVQSGAEVKKPGASVKVSCKVSGFSFPSSGVDWVRQAPGQGLEWLGVTW |
| | | GVGSTHYNQKFQGRVTITKDTSKSTVYMELSSLRSEDTAVYYCASGEWDEGFVYWGQ |
| | | GTLVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEPKSC |
| | | DKTHTCPPCPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| 31 | DNA | GATATTCAGCTGACCCAGAGCCCATCCTTCCTCAGCGCCTCCGTGGGGGATAGAGTGACCATTACATGT |
| | GBT-1765 LC | AAGGCCTCACAGAATGTAGGGACTGCCGTCGCTTGGTACCAGCAGAAGCCCGGCAAGGCACCAAAGCT |
| | GBT-2053 LC | CCTGATTTATGCCGCTTCCACTCTGCAGAGTGGTGTCCCCAGCAGATTCTCAGGTTCTGGCTCTGGAACG |
| | | GAATTCACCCTGACCATCTCTTCTCTGCAACCCGAAGACTTTGCTACCTACTATTGTCAACAGTATAGCTC |
| | | ATACCCACTGACCTTCGGTGGAGGAACGAAAGTTGAGATTAAACGTACGGTGGCTGCACCATCTGTCTT |
| | | CATCTTCCCGCCATCTGATGAGCAGTTGAAATCTGGAACTGCCTCTGTTGTGTGCCTGCTGAATAACTTC |
| | | TATCCCAGAGAGGCCAAAGTACAGTGGAAGGTGGATAACGCCCTCCAATCGGGTAACTCCCAGGAGAG |
| | | TGTCACAGAGCAGGACAGCAAGGACAGCACCTACAGCCTCAGCAGCACCCTGACGCTGAGCAAAGCAG |
| | | ACTACGAGAAACACAAAGTCTACGCCTGCGAAGTCACCCATCAGGGCCTGAGCTCGCCCGTCACAAAGA |
| | | GCTTCAACAGGGGAGAGTGT |
| 32 | DNA | CAGGTTCAGCTTGTGCAAAGCGGCGCAGAGGTCAAGAAGCCAGGGGCCTCCGTCAAGGTGTCCTGTAA |
| | GBT-1765 HC | AGTCTCTGGCTTTTCCTTCCCATCCTCTGGGGGGACTGGGTCCGCCAGGCTCCAGGGCAAGGGCTCGA |
| | | GTGGTTGGGCGTTACATGGGGAGTGGGCTCCACCCACTACAATCAAAAGTTCCAGGGCCGTGTTACAAT |
| | | CACCAAAGATACCTCTAAAAGCACTGTGTACATGGAGCTGTCATCATTGAGGTCCGAAGATACCGCCGT |
| | | TTATTACTGTGCATCAGGGGAGTGGGACGAGGGCTTTGTTTATTGGGGGCAGGGAACCTTGGTAACAG |
| | | TCTCAAGCGCGTCGACCAAGGGCCCATCGGTCTTCCCCCTGGCACCCTCCTCCAAGAGCACCTCTGGGG |
| | | GCACAGCGGCCCTGGGCTGCCTGGTCAAGGACTACTTCCCCGAACCGGTGACGGTGTCGTGGAACTCA |
| | | GGCGCCCTGACCAGCGGCGTGCACACCTTCCCGGCTGTCCTACAGTCCTCAGGACTCTACTCCCTCAGCA |
| | | GCGTGGTGACCGTGCCCTCCAGCAGCTTGGGCACCCAGACCTACATCTGCAACGTGAATCACAAGCCCA |
| | | GCAACACCAAGGTGGACAAGAAAGTTGAGCCCAAATCTTGTGACAAAACTCACACATGCCCACCGTGCC |
| | | CAGCACCTGAAGCCGCTGGGGCACCGTCAGTCTTCCTCTTCCCCCCAAAACCCAAGGACACCCTCATGAT |
| | | CTCCCGGACCCCTGAGGTCACATGCGTGGTGGTGGACGTGAGCCACGAAGACCCTGAGGTCAAGTTCA |
| | | ACTGGTACGTGGACGGCGTGGAGGTGCATAATGCCAAGACAAAGCCGCGGGAGGAGCAGTACAACAG |
| | | CACGTACCGTGTGGTCAGCGTCCTCACCGTCCTGCACCAGGACTGGCTGAATGGCAAGGAGTACAAGT |
| | | GCAAGGTCTCCAACAAAGCCCTCCCAGCCCCCATCGAGAAAACCATCTCCAAAGCCAAAGGGCAGCCCC |
| | | GAGAACCACAGGTGTACACCCTGCCCCCATCCCGGGAGGAGATGACCAAGAACCAGGTCAGCCTGACC |
| | | TGCCTGGTCAAAGGCTTCTATCCCAGCGACATCGCCGTGGAGTGGGAGAGCAATGGGCAGCCGGAGAA |
| | | CAACTACAAGACCACGCCTCCCGTGCTGGACTCCGACGGCTCCTTCTTCCTCTATAGCAAGCTCACCGTG |
| | | GACAAGAGCAGGTGGCAGCAGGGGAACGTCTTCTCATGCTCCGTGATGCATGAGGCTCTGCACAACCA |
| | | CTACACGCAGAAGAGCCTCTCCCTGTCCCCGGGAAAA |
| 33 | DNA | CAAGTCCAGCTTGTGCAGAGCGGTGCTGAGGTTAAAAAGCCTGGGGCAAGTGTCAAAGTCAGCTGTAA |
| | GBT-2053 dual Fab | AGTCAGCGGTTTTAGCTTTCCGTCCTCAGGCGTGGACTGGGTCAGACAGGCCCCTGGTCAAGGCCTCGA |
| | HC | ATGGCTCGGTGTGACATGGGGTGTGGGCTCCACCCATTACAATCAGAAATTTCAGGGAAGAGTTACAAT |
| | | CACGAAGGACACTAGTAAATCAACAGTGTACATGGAGTTGAGTAGCCTTCGCAGCGAGGACACGGCCG |
| | | TCTACTACTGCGCCAGTGGAGAGTGGGACGAGGGTTTTGTATATTGGGGACAGGGAACTCTCGTGACT |
| | | GTGAGTTCCGCCTCGACCAAGGGCCCATCGGTCTTCCCCCTGGCACCCTCCTCCAAGAGCACCTCTGGG |
| | | GGCACAGCGGCCCTGGGCTGCCTGGTCAAGGACTACTTCCCCGAACCGGTGACGGTGTCGTGGAACTC |
| | | AGGCGCCCTGACCAGCGGCGTGCACACCTTCCCGGCTGTCCTACAGTCCTCAGGACTCTACTCCCTCAGC |
| | | AGCGTGGTGACCGTGCCCTCCAGCAGCTTGGGCACCCAGACCTACATCTGCAACGTGAATCACAAGCCC |
| | | AGCAACACCAAGGTGGACAAGAAAGTTGAGCCCAAATCTTGT [symbol] CAGGTTCAGCT |
| | | TGTGCAAAGCGGCGCAGAGGTCAAGAAGCCAGGGGCCTCCGTCAAGGTGTCCTGTAAAGTCTCTGGCT |
| | | TTTCCTTCCCATCCTCTGGGGTGGACTGGGTCCGCCAGGCTCCAGGGCAAGGGCTCGAGTGGTTGGGC |
| | | GTTACATGGGGAGTGGGCTCCACCCACTACAATCAAAAGTTCCAGGGCCGTGTTACAATCACCAAAGAT |
| | | ACCTCTAAAAGCACTGTGTACATGGAGCTGTCATCATTGAGGTCCGAAGATACCGCCGTTTATTACTGTG |
| | | CATCAGGGGAGTGGGACGAGGGCTTTGTTTATTGGGGGCAGGGAACCTTGGTAACAGTCTCAAGCGCG |
| | | TCGACCAAGGGCCCATCGGTCTTCCCCCTGGCACCCTCCTCCAAGAGCACCTCTGGGGGCACAGCGGCC |
| | | CTGGGCTGCCTGGTCAAGGACTACTTCCCCGAACCGGTGACGGTGTCGTGGAACTCAGGCGCCCTGACC |
| | | AGCGGCGTGCACACCTTCCCGGCTGTCCTACAGTCCTCAGGACTCTACTCCCTCAGCAGCGTGGTGACC |
| | | GTGCCCTCCAGCAGCTTGGGCACCCAGACCTACATCTGCAACGTGAATCACAAGCCCAGCAACACCAAG |
| | | GTGGACAAGAAAGTTGAGCCCAAATCTTGTGACAAAACTCACACATGCCCACCGTGCCCAGCACCTGAA |
| | | GCCGCTGGGGCACCGTCAGTCTTCCTCTTCCCCCCAAAACCCAAGGACACCCTCATGATCTCCCGGACCC |
| | | CTGAGGTCACATGCGTGGTGGTGGACGTGAGCCACGAAGACCCTGAGGTCAAGTTCAACTGGTACGTG |
| | | GACGGCGTGGAGGTGCATAATGCCAAGACAAAGCCGCGGGAGGAGCAGTACAACAGCACGTACCGTG |
| | | TGGTCAGCGTCCTCACCGTCCTGCACCAGGACTGGCTGAATGGCAAGGAGTACAAGTGCAAGGTCTCC |
| | | AACAAAGCCCTCCCAGCCCCCATCGAGAAAACCATCTCCAAAGCCAAAGGGCAGCCCCGAGAACCACA |
| | | GGTGTACACCCTGCCCCCATCCCGGGAGGAGATGACCAAGAACCAGGTCAGCCTGACCTGCCTGGTCA |
| | | AAGGCTTCTATCCCAGCGACATCGCCGTGGAGTGGGAGAGCAATGGGCAGCCGGAGAACAACTACAA |
| | | GACCACGCCTCCCGTGCTGGACTCCGACGGCTCCTTCTTCCTCTATAGCAAGCTCACCGTGGACAAGAGC |
| | | AGGTGGCAGCAGGGGAACGTCTTCTCATGCTCCGTGATGCATGAGGCTCTGCACAACCACTACACGCA |
| | | GAAGAGCCTCTCCCTGTCCCCGGGAAAA |
| 34 | Human TNFR2 | MAPVAVWAALAVGLELWAAAHALPAQVAFTPYAPEPGSTCRLREYYDQTAQMCCSKCSPGQHAK |
| | | VFCTKTSDTVCDSCEDSTYTQLWNWVPECLSCGSRCSSDQVETQACTREQNRICTCRPGWYCAL |
| | | SKQEGCRLCAPLRKCRPGFGVARPGTETSDVVCKPCAPGTFSNTTSSTDICRPHQICNVVAIPG |
| | | NASMDAVCTSTSPTRSMAPGAVHLPQPVSTRSQHTQPTPEPSTAPSTSFLLPMGPSPPAEGSTG |
| | | DFALPVGLIVGVTALGLLIIGVVNCVIMTQVKKKPLCLQREAKVPHLPADKARGTQGPEQQHLL |
| | | ITAPSSSSSSLESSASALDRRAPTRNQPQAPGVEASGAGEARASTGSSDSSPGGHGTQVNVTCI |
| | | VNVCSSSDHSSQCSSQASSTMGDTDSSPSESPKDEQVPFSKEECAFRSQLETPETLLGSTEEKP |
| | | LPLGVPDAGMKPS |
| 35 | Murine TNFR2 | MAPAALWVALVFELQLWATGHTVPAQVVLTPYKPEPGYECQISQEYYDRKAQMCCAKCPPGQYV |
| | | KHFCNKTSDTVCADCEASMYTQVWNQFRTCLSCSSSCTTDQVEIRACTKQQNRVCACEAGRYCA |
| | | LKTHSGSCRQCMRLSKCGPGFGVASSRAPNGNVLCKACAPGTFSDTTSSTDVCRPHRICSILAI |
| | | PGNASTDAVCAPESPTLSAIPRTLYVSQPEPTRSQPLDQEPGPSQTPSILTSLGSTPIIEQSTK |
| | | GGISLPIGLIVGVTSLGLLMLGLVNCIILVQRKKKPSCLQRDAKVPHVPDEKSQDAVGLEQQHL |
| | | LTTAPSSSSSSLESSASAGDRRAPPGGHPQARVMAEAQGFQEARASSRISDSSHGSHGTHVNVT |
| | | CIVNVCSSSDHSSQCSSQASATVGDPDAKPSASPKDEQVPFSQEECPSQSPCETTETLQSHEKP |
| | | LPLGVPDMGMKPSQAGWFDQIAVKVA |
| 36 | Cynomolgus TNFR2 | MAPAAVWAALAVGLELWAAGHALPAQVAFTPYAPEPGGTCRLREYYDQTAQMCCSKCPPGQHAK |
| | | VFCTKTSDTVCDSCEDSTYTQLWNWVPECLSCGSRCSSDQVETQACTREQNRICTCRPGWYCAL |
| | | SKQEGCRLCAQLRKCRPGFGVARPGTETSDVVCKPCAPGTFSNTTSSTDICRPHQICHVVAIPG |
| | | NASMDAVCTSTSPTRSMAPGAVHLPQPVSTRSQHTQPTPAPSTAPGTSFLLPVGPSPPAEGSTG |
| | | DIVLPVGLIVGVTALGLLIIGVVNCVIMTQVKKKPLCLQRETKVPHLPADKARGAQGPEQQHLL |
| | | TTVPSSSSSSLESSASALDRRAPTRNQPQAPGAEKASGAGEARASTGSSADSSPGGHGTQVNVT |
| | | CIVNVCSSSDHSSQCSSQASSTMGDTDASPSGSPKDEQVPFSKEESAFRSQLETPETLLGSTEE |
| | | KPLPLGVPDAGMKPS |
| 37 | Fc chain of 2053 | DKTHTCPPCPAPEAAGAPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |

1. An isolated antibody that specifically binds to TNFR2, a heavy chain variable region (VH) and a light chain variable region (VL), comprising one or more selected from the group consisting of
(i) comprising the CDR-H1, CDR-H2, and CDR-H3 sequences of a VH sequence selected from the group consisting of SEQ ID NO: 30, SEQ ID NO: 13, SEQ ID NO: 19, SEQ ID NO: 21, SEQ ID NO: 22, and SEQ ID NO: 29; and the CDR-L1, CDR-L2, and CDR-L3 sequences of a VL sequence selected from the group consisting of SEQ ID NO: 9, SEQ ID NO: 6, SEQ ID NO: 8, and SEQ ID NO: 4;
(ii) a CDR-L1 sequence according to SEQ ID NO: 1; a CDR-L2 sequence according to SEQ ID NO: 7, and a CDR-L3 sequence according to SEQ ID NO: 3, and a CDR-H1 sequence according to SEQ ID NO: 10; a CDR-H2 sequence according to SEQ ID NO: 20; a CDR-H3 sequence according to SEQ ID NO: 12; and
(iii) a CDR-L1 sequence according to SEQ ID NO: 1; a CDR-L2 sequence according to SEQ ID NO: 2, and a CDR-L3 sequence according to SEQ ID NO: 3, and a CDR-H1 sequence according to SEQ ID NO: 10; a CDR-H2 sequence according to SEQ ID NO: 11; a CDR-H3 sequence according to SEQ ID NO: 12.

2. The antibody of claim 1, comprising one or both of
(i) a VH framework sequence derived from a human germline VH sequence selected from the group consisting of IGHV1-46, IGHV4-31, IGHV4-30-4, and IGHV4-4; and
(ii) a VL framework sequence derived from a human germline VL sequence selected from the group consisting of IGKV1-9, IGKV1-33, IGKV1-27, IGKV1-39, IGKV1-9, IGKV1-1, and IGKV1-11.

3. The antibody of claim 1, wherein the VL comprises an amino acid sequence according to a sequence selected from the group consisting of SEQ ID NO: 8 and SEQ ID NO: 4, and wherein the VH comprises an amino acid sequence according to a sequence selected from the group consisting of SEQ ID NO: 21 and SEQ ID NO: 13.

4. The antibody of claim 1, comprising the VH sequence of SEQ ID NO: 21, and the VL of SEQ ID NO: 8.

5. The antibody of claim 1, further comprising an Fc domain, wherein the Fc domain is the Fc domain of an IgA, IgD, IgE, IgM, or IgG.

6. The antibody of claim 1, further comprising one or both of a heavy chain (HC) comprising a sequence in accordance with SEQ ID NO: 22, And a light chain (LC) comprising a sequence in accordance with SEQ ID NO: 9.

7. The antibody of claim 1, comprising a first Fab, second Fab, third Fab, and fourth Fab wherein the first Fab, second Fab, third Fab, and fourth Fab each comprise a CDR-L1 sequence according to SEQ ID NO: 1; a CDR-L2 sequence according to SEQ ID NO: 7, and a CDR-L3 sequence according to SEQ ID NO: 3, and a CDR-H1 sequence according to SEQ ID NO: 10; a CDR-H2 sequence according to SEQ ID NO: 20; a CDR-H3 sequence according to SEQ ID NO: 12.

8. The antibody of claim 7, wherein the first Fab, second Fab, third Fab, and fourth Fab each comprise a VH with a sequence according to SEQ ID NO: 21 and a VL with a sequence according to SEQ ID NO: 8.

9. The antibody of claim 7, wherein the N′ terminus of the first Fab is connected to the C′-terminus of the third Fab via a first linker, and the N′ terminus of the second Fab is connected to the C′-terminus of the fourth Fab via a second linker, and wherein optionally, one or both of the first linker and the second linker comprises a sequence in accordance with SEQ ID NO: 27.

10. The antibody of claim 1, comprising a heavy chain (HC) comprising a sequence according to SEQ ID NO: 30, and a light chain (LC), comprising a sequence according to SEQ ID NO: 9.

11. The antibody of claim 1, comprising a heavy chain (HC) sequence encoded by a nucleic acid sequence of SEQ ID NO: 33, and a light chain (LC) sequence encoded by a nucleic acid sequence of SEQ ID NO: 31.

12. An antibody comprising
(i) one or both of a VH sequence encoded by the plasmid deposited at the ATCC and having ATCC Accession No. PTA-127528 and a VH sequence encoded by the plasmid deposited at the ATCC and having ATCC Accession No. PTA-127529; and a VL sequence encoded by the plasmid deposited at the ATCC and having ATCC Accession No. PTA-127531; or
(ii) a HC sequence encoded by the plasmid deposited at the ATCC and having ATCC Accession No. PTA-127530 and a LC sequence encoded by the plasmid deposited at the ATCC and having ATCC Accession No. PTA-127532.

13. The antibody of claim 1, wherein the antibody is characterized by one or more selected from the group consisting of:
an EC₅₀ of less than 5 μM in a human TNFR2 potency assay in Jurkat reporter cells;
(ii) an EC₅₀ of less than 10 μM in a human TNFR2 potency assay in human peripheral blood monocytes;
(iii) an EC₅₀ of less than 5 μM in a human TNFR2 potency assay in Jurkat reporter cells;
(iv) an EC₅₀ of less than 2 mg/ml for ICAM-1 upregulation in human TNFR2 expressing primary T-cell population from human peripheral blood monocytes;
(v) an EC₅₀ of less than 1 mg/ml for ICAM-1 upregulation in human TNFR2 expressing primary T-cell population from human peripheral blood monocytes; and
(vi) an affinity KD for human TNFR2 of less than 1 nm.

14. The antibody of claim 1, for use as a medicament.

15. The antibody of claim 14, wherein the use is for the treatment of one or more selected from the group consisting of rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE).

16. A pharmaceutical composition comprising a therapeutically effective amount of the antibody of claim 1 and a pharmaceutically acceptable carrier.

17. A method of treating a medical condition, comprising administering to a subject in need thereof a therapeutically effective amount of the pharmaceutical composition of claim 16.

18. The method of claim 17, wherein the condition is selected from the group consisting of rheumatoid arthritis (RA), ankylosing spondylitis (AS), Chron's disease (CD), ulcerative colitis (UC), graft-versus-host-disease (GVHD), transplantation, psoriasis (PSO), psoriatic arthritis (PSA), atopic dermatitis (AD), vitiligo, alopecia areata (AA), type 1 diabetes (T1D), multiple sclerosis (MS), irritable bowel disease (IBD), autoimmune hepatitis and systemic erythematosus lupus (SLE).

19. An isolated polynucleotide, comprising one or more nucleotide sequences encoding the antibody of claim 1.

20. An isolated polynucleotide encoding one or more of
(i) a HC and a LC, or both, of an antibody that binds to TNFR2, wherein said nucleic acid comprises one or more selected from the group consisting of the nucleic acid sequence of SEQ ID NO: 31, the nucleic acid sequence of SEQ ID NO: 32, the nucleic acid sequence of SEQ ID NO: 33;
(ii) an isolated antibody that specifically binds to TNFR2, comprising a heavy chain (HC) sequence encoded by a nucleic acid sequence of SEQ ID NO: 33, and a light chain (LC) sequence encoded by a nucleic acid sequence of SEQ ID NO: 31;
(iii) an isolated antibody comprising a HC sequence encoded by the plasmid deposited at the ATCC and having ATCC Accession No. PTA-127530 and a LC sequence encoded by the plasmid deposited at the ATCC and having ATCC Accession No. PTA-127532.

21. A vector comprising the polynucleotide of claim 19.

22. An isolated host cell comprising the polynucleotide of claim 1.

23. A method of producing an isolated antibody, comprising culturing the host cell of claim 22 under conditions that result in production of the antibody, and recovering the antibody.


SEQUENCE LISTING (37 sequences)

SEQ ID NO: 1 - protein, 11 aa, synthetic construct
KASQNVGTAVA

SEQ ID NO: 2 - protein, 7 aa, synthetic construct
SASNRYT

SEQ ID NO: 3 - protein, 9 aa, synthetic construct
QQYSSYPLT

SEQ ID NO: 4 - protein, 107 aa, synthetic construct
DIVMTQSQKFMSTTVGDRVSITCKASQNVGTAVAWYQQKPGQSPKLLIYSASNRYTGVPDRFTGSGSGTDFTLTISNMQSEDLADYFCQQYSSYPLTFGGGTKLEIK

SEQ ID NO: 5 - protein, 107 aa, synthetic construct [sequence omitted]

SEQ ID NO: 6 - protein, 214 aa, synthetic construct
DIVMTQSQKFMSTTVGDRVSITCKASQNVGTAVAWYQQKPGQSPKLLIYSASNRYTGVPDRFTGSGSGTDFTLTISNMQSEDLADYFCQQYSSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 7 aa, synthetic construct
AASTLQS

SEQ ID NO: 8 - protein, 107 aa, synthetic construct
DIQLTQSPSFLSASVGDRVTITCKASQNVGTAVAWYQQKPGKAPKLLIYAASTLQSGVPSRFSGSGSGTEFTLTISSLQPEDFATYYCQQYSSYPLTFGGGTKVEIK

SEQ ID NO: 9 - protein, 215 aa, synthetic construct
DIQLTQSPSFLSASVGDRVTITCKASQNVGTAVAWYQQKPGKAPKLLIYAASTLQSGVPSRFSGSGSGTEFTLTISSLQPEDFATYYCQQYSSYPLTFGGGTKVEIKRTVAAPSVFIFPPSDEQLKSGVTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 10 aa, synthetic construct
GFSFPSSGVD

SEQ ID NO: 11 - protein, 16 aa, synthetic construct
VTWGVGSTHYNSALKS

SEQ ID NO: 12 - protein, 9 aa, synthetic construct
GEWDEGFVY

SEQ ID NO: 13 - protein, 117 aa, synthetic construct
QVQLKESGPGLVAPSQSLSITCTVSGFSFPSSGVDWVRQSPGKGLEWLGVTWGVGSTHYNSALKSRLSISKDNSKSQVFLKMNSLQTEDTAIYYCASGEWDEGFVYWGQGTLVTVSA

SEQ ID NO: 14 - protein, 98 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 15 - protein, 12 aa, synthetic construct
ERKCCVECPPCP

SEQ ID NO: 16 - protein, 109 aa, synthetic construct
APPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTK

SEQ ID NO: 17 - protein, 107 aa, synthetic construct
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 228 aa, synthetic construct
ERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 443 aa, synthetic construct
QVQLKESGPGLVAPSQSLSITCTVSGFSFPSSGVDWVRQSPGKGLEWLGVTWGVGSTHYNSALKSRLSISKDNSKSQVFLKMNSLQTEDTAIYYCASGEWDEGFVYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 16 aa, synthetic construct
VTWGVGSTHYNQKFQG

SEQ ID NO: 21 - protein, 117 aa, synthetic construct
QVQLVQSGAEVKKPGASVKVSCKVSGFSFPSSGVDWVRQAPGQGLEWLGVTWGVGSTHYNQKFQGRVTITKDTSKSTVYMELSSLRSEDTAVYYCASGEWDEGFVYWGQGTLVTVSS

SEQ ID NO: 22 - protein, 443 aa, synthetic construct
QVQLVQSGAEVKKPGASVKVSCKVSGFSFPSSGVDWVRQAPGQGLEWLGVTWGVGSTHYNQKFQGRVTITKDTSKSTVYMELSSLRSEDTAVYYCASGEWDEGFVYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 24 - protein, 10 aa, synthetic construct
DKTHTCPPCP

SEQ ID NO: 25 - protein, 110 aa, synthetic construct
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 26 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 5 aa, synthetic construct
GGGGS

SEQ ID NO: 28 - protein, 108 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGS

SEQ ID NO: 29 - protein, 342 aa, synthetic construct
QVQLVQSGAEVKKPGASVKVSCKVSGFSFPSSGVDWVRQAPGQGLEWLGVTWGVGSTHYNQKFQGRVTITKDTSKSTVYMELSSLRSEDTAVYYCASGEWDEGFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQVQLVQSGAEVKKPGASVKVSCKVSGFSFPSSGVDWVRQAPGQGLEWLGVTWGVGSTHYNQKFQGRVTITKDTSKSTVYMELSSLRSEDTAVYYCASGEWDEGFVYWGQGTLVTVSS

SEQ ID NO: 30 - protein, 672 aa, synthetic construct
QVQLVQSGAEVKKPGASVKVSCKVSGFSFPSSGVDWVRQAPGQGLEWLGVTWGVGSTHYNQKFQGRVTITKDTSKSTVYMELSSLRSEDTAVYYCASGEWDEGFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQVQLVQSGAEVKKPGASVKVSCKVSGFSFPSSGVDWVRQAPGQGLEWLGVTWGVGSTHYNQKFQGRVTITKDTSKSTVYMELSSLRSEDTAVYYCASGEWDEGFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 642 bp, synthetic construct
  source 1..642: mol_type = other DNA
gatattcagctgacccagagcccatccttcctcagcgcctccgtgggggatagagtgaccattacatgtaaggcctcacagaatgtagggactgccgtcgcttggtaccagcagaagcccggcaaggcaccaaagctcctgatttatgccgcttccactctgcagagtggtgtccccagcagattctcaggttctggctctggaacggaattcaccctgaccatctcttctctgcaacccgaagactttgctacctactattgtcaacagtatagctcatacccactgaccttcggtggaggaacgaaagttgagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 32 - DNA, 1341 bp, synthetic construct
  source 1..1341: mol_type = other DNA
caggttcagcttgtgcaaagcggcgcagaggtcaagaagccaggggcctccgtcaaggtgtcctgtaaagtctctggcttttccttcccatcctctggggtggactgggtccgccaggctccagggcaagggctcgagtggttgggcgttacatggggagtgggctccacccactacaatcaaaagttccagggccgtgttacaatcaccaaagatacctctaaaagcactgtgtacatggagctgtcatcattgaggtccgaagataccgccgtttattactgtgcatcaggggagtgggacgagggctttgtttattgggggcagggaaccttggtaacagtctcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggaaaa

SEQ ID NO: 33 - DNA, 2016 bp, synthetic construct
  source 1..2016: mol_type = other DNA
caagtccagcttgtgcagagcggtgctgaggttaaaaagcctggggcaagtgtcaaagtcagctgtaaagtcagcggttttagctttccgtcctcaggcgtggactgggtcagacaggcccctggtcaaggcctcgaatggctcggtgtgacatggggtgtgggctccacccattacaatcagaaatttcagggaagagttacaatcacgaaggacactagtaaatcaacagtgtacatggagttgagtagccttcgcagcgaggacacggccgtctactactgcgccagtggagagtgggacgagggttttgtatattggggacagggaactctcgtgactgtgagttccgcctcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtggcggtggcgggtcccaggttcagcttgtgcaaagcggcgcagaggtcaagaagccaggggcctccgtcaaggtgtcctgtaaagtctctggcttttccttcccatcctctggggtggactgggtccgccaggctccagggcaagggctcgagtggttgggcgttacatggggagtgggctccacccactacaatcaaaagttccagggccgtgttacaatcaccaaagatacctctaaaagcactgtgtacatggagctgtcatcattgaggtccgaagataccgccgtttattactgtgcatcaggggagtgggacgagggctttgtttattgggggcagggaaccttggtaacagtctcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggaaaa

SEQ ID NO: 34 - protein, 461 aa, synthetic construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 35 - protein, 474 aa, synthetic construct
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 36 - protein, 463 aa, synthetic construct
MAPAAVWAALAVGLELWAAGHALPAQVAFTPYAPEPGGTCRLREYYDQTAQMCCSKCPPGQHAKVFCTKTSDTVCDSCEDSTYTQLWNWVPECLSCGSRCSSDQVETQACTREQNRICTCRPGWYCALSKQEGCRLCAQLRKCRPGFGVARPGTETSDVVCKPCAPGTFSNTTSSTDICRPHQICHVVAIPGNASMDAVCTSTSPTRSMAPGAVHLPQPVSTRSQHTQPTPAPSTAPGTSFLLPVGPSPPAEGSTGDIVLPVGLIVGVTALGLLIIGVVNCVIMTQVKKKPLCLQRETKVPHLPADKARGAQGPEQQHLLTTVPSSSSSSLESSASALDRRAPTRNQPQAPGAEKASGAGEARASTGSSADSSPGGHGTQVNVTCIVNVCSSSDHSSQCSSQASSTMGDTDASPSGSPKDEQVPFSKEESAFRSQLETPETLLGSTEEKPLPLGVPDAGMKPS

SEQ ID NO: 37 - protein, 227 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK